SEQUENCE LISTING (10 sequences)

SEQ ID NO: 1 - protein, 268 aa, Thermomyces lanuginosus
EVSQDLFNQFNLFAQYSAAAYCGKNNRAPAGTNITCTANACPEVEKADATFLYSFEDSGVGDVTGFLALDNTNKLIVLSFRGSRSIENWIGNLNFELKEINDICSGCRGHAGFTSSWRSVADTLRQKVEDAVREHPDYRVVFTGHSLGGALATVAGADLRGNKYDIDVFSYGAPRVGNRAFAEFLTVQTGGTLYRITHTNDIVPRLPPREFGYSHSSPEYWISGTLVPVRRRDIVKIEGIDATGGNNQPNIPSITAHLWYFGLIGTCL

SEQ ID NO: 2 - protein, 274 aa, Thermomyces lanuginosus
SPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVTRNDIVKIEGIDATGGNNQPNIPDIPAHLWYFGLIGTCL

SEQ ID NO: 3 - protein, 323 aa, Moesziomyces antarcticus
TPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 4 - protein, 319 aa, Chromobacterium viscosum
ADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV

SEQ ID NO: 5 - protein, 395 aa, Aneurinibacillus thermoaerophilus
AVDSNHTPAAPELVARKNQYPIVLVHGFAGWGRDEMLGVKYWGGMHDIQEDLKQYGYETHTAVVGPFSSNWDRACELYAQLVGGTVDYGAAHAEKYGHDRFGRTYPGLLKNWDGEHKIHLIGHSMGGQTVRVLTQLLKEGSQEEREYAKKHGVQLSPLFEGGKSWVHSVTTIATPNDGTTLADVVTQLIPAAQQIMGLAAAVSGNTNVPVYDFKLDQWGLKRKAGESFVHYADRVWNSGIWTNTKDISAWDLKPEGAKELNNWVKAQPDVYYFSYSGEATFRSLITGHHLPDLTMNKLITPFGIFLGCYGSDEKWWQNDGIVNTISMNGPKLGSTDEIVPYDGTPKIGKWNDMGIQENWDHADYIGLSLSYVLGIEKIEDFYRGVADMLGSLSVR

SEQ ID NO: 6 - protein, 388 aa, Geobacillus sp.
ASPRANDAPIVLLHGFTGWGREEMFGFKYWGGVRGDIEQWLNDNGYQAYTLAVGPLSSNWDRACEAYAQLVGGTVDYGAAHAAKHGHARFGRTYPGLLPELKRGGRIHIIAHSQGGQTARMLVSLLENGSQEEREYAKEHNVSLSPLFEGGHRFVLSVTTIATPHDGTTLVNMVDFTDRFFDLQKAVLEAAAVASNAPYTSEIYDFKLDQWGLRREPGESFDHYFERLKRSPVWTSTDTARYDLSVPGAETLNRWVKASPNTYYLSFSTERTYRGALTGNYYPELGMNAFSAIVCAPFLGSYRNAALGIDSHWLENDGIVNTISMNGPKRGSNDRIVPYDGTLKKGVWNDMGTCNVDHLEVIGVDPNPSFNIRAFYLRLAEQLASLRP

SEQ ID NO: 7 - protein, 319 aa, Burkholderia glumae; identical to SEQ ID NO: 4

SEQ ID NO: 8 - protein, 274 aa, Thermomyces lanuginosus
SPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 9 - protein, 269 aa, Thermomyces lanuginosus
CVSQDLFNQFNLFAQYSAAAYCGKNNRAPAGTNITCTANACPEVEKADATVLYSFEDSGVGDVTGFLALDNTNKLIVLSFRGSRSIENWIGNLNFELIEINDICSGCRGHAGFTSSWRSVADTLRQKVEDAVREHPDYRVVFTGHSLGGALATVAGADLRGNKYDIDVFSYGAPRVGNRAFAEFLTVQTGGTLYRITSTNDIVPRLPPREFGYSHSSPEFWIKSGTLVPVRRCDIVKIEGIDATGGNNQPNIPSITAHLWYFGLIGTCL

SEQ ID NO: 10 - protein, 274 aa, Thermomyces lanuginosus
SPIRREVSQDLFNQFNLFAQYSAAAYCGKNNRAPAGTKITCTCNACPEVEKADATFLYSFEDSGVGDVTGFLALDNTNKLIVLSFRGSRSIENWIGNLNFRLKEINDICSGCRGHAGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVERRDIVKIEGIDATGGNNQPNIPDIPAHLWYFGLIGTCL

(19) United States
(12) Patent Application Publication
(10) Pub. No.: US 2025/0263755 A1
(43) Pub. Date: Aug. 21, 2025

(54) PROCESS FOR REDUCING FREE FATTY ACIDS

(71) Applicant: Novozymes A/S, Bagsvaerd (DK)

(72) Inventors: Rasmus Boeg HANSEN, Frederiksberg (DK); Johan MOGENSEN, Gentofte (DK)

(73) Assignee: Novozymes A/S, Bagsvaerd (DK)

(21) Appl. No.: 18/857,422

(22) PCT Filed: May 16, 2023
(86) PCT No.: PCT/EP2023/063046

§ 371 Date: Oct. 16, 2024

(30) Foreign Application Priority Data
May 17, 2022 (EP) 22173875.0
Apr. 27, 2023 (EP) 23170320.8

(51) Int. Cl. C12P 7/04; C10G 29/20; C12N 9/18

(52) U.S. Cl. CPC C12P 7/04; C10G 29/20; C12N 9/18; C12Y 301/01001; C10G 2400/04

(57) ABSTRACT

The present invention relates to a process for enzymatic (trans)esterification/esterification of free fatty acids and glycerides. In particular, the invention relates to this process using a drying operation for water removal from enzyme reaction mixture continuously or by separating the glycerol phase from the reaction mixture, then drying the glycerol phase and recirculating to reform the reaction mixture, especially in biodiesel applications, which facilitates reduction of FFA in the biodiesel.

### FIELD OF THE INVENTION

The present invention relates to a process for reducing the level of free fatty acids in biodiesel/fatty acid alkyl esters by enzymatic combined (trans)esterification/esterification reaction. In particular, the invention relates to this process using low water content and recirculation of glycerol in the combined (trans)esterification/esterification reaction.

### BACKGROUND OF THE INVENTION

CO₂ stemming from fossil fuels causes environmental problems, and this forces the world to seek sustainable solutions.

Conventional diesel fuel stemming from fossil carbon sources is currently a major contributor to the CO₂ emissions, and the development of sustainable and renewable fuels capable of driving existing diesel motors would be a possible way of lowering the diesel related CO₂ emissions. Such a fuel is for instance fatty acid methyl esters also called biodiesel, which is obtainable through the esterification of fatty acids originating from oils and fats consisting of glycerides and free fatty acids (FFA).

The biomass feedstocks for biodiesel production consist mainly of glycerides, but cheap, low quality feedstocks often also hold significant amounts of free fatty acids (FFA). However, FFA are also a potential source of biodiesel as they are convertible into biodiesel through (trans)esterification.

Traditionally, production of biodiesel has been done through chemical catalysis. Alkaline-catalyzed biodiesel production through transesterification of glycerides is the most commonly used method but has drawbacks. Importantly, chemical alkaline catalysis is unable to handle low and varying feedstock qualities where the concentration of FFA is significant. In order to exploit such feedstocks, extensive pretreatment is required. Today, a typical pretreatment is sulfuric acid catalyzed esterification of FFA.

The requirement for extensive pre-processing is a problem as this greatly reduces the profitability of the process. To obtain a greener, more profitable, and more robust process in terms of usability of low-quality feedstocks, enzymatic catalysis offers a solution.

Enzymatic catalysis is less sensitive to feedstock qualities. It requires operating conditions much closer to ambient, and results in smaller, cleaner, and more easily disposable waste streams because of high selectivity and low additive requirements. Additionally, enzymes are renewable catalysts and processes utilizing enzymes tend to be greener than chemical processes.

Enzymatic biodiesel processes require only minor pre-processing relative to the chemical processes and are able to convert both FFA and glycerides to biodiesel.

However, enzymatic processes do have some limitations. The enzymatic esterification reaction of free fatty acids with short chain alcohols to biodiesel and water, is reversible. As such, a presence of water will drive the reaction in the direction which is unfavourable to biodiesel production. At the same time, the removal of water which would favour the formation of biodiesel, would have the negative effect of decreasing enzyme stability.

Although the current enzymatic transesterification process yields high conversion, the product still contains 1-6 wt % FFA (regardless of feedstock) due to the unavoidable equilibrium constraint. One solution could be to use a caustic wash to convert and remove the residual FFA, but because it produces soap, this requires extra processing with significant amounts of wastewater as byproduct.

Thus, there is still a need for development of enzymatic process for biodiesel production, in particular where the efficiency of the enzymatic process is increased.

### SUMMARY OF THE INVENTION

The invention relates to a process for reducing level of free fatty acids in biodiesel/fatty acid alkyl esters, said process comprising steps of: (i) providing a fatty acid feedstock substrate comprising triglycerides, diglycerides, monoglycerides, free fatty acids, fatty acid esters, or any combination thereof; (ii) reacting said fatty acid feedstock substrate with alcohol in the presence of one or more esterases and added glycerol to produce fatty acid alkyl esters, (iii) separating the reaction mixture of step (ii) into light phase comprising fatty acid methyl ester (FAME) and a heavy phase comprising esterase, glycerol, short chain alcohol and water; (iv) drying the mixture of step ii) followed by separation of the light and heavy phases; and/or (v) drying the heavy phase of step (iii) in presence of the esterase, wherein the glycerol and esterase of the heavy phase is recycled and added back to step (ii); and wherein the resulting level of free fatty acid (FFA) concentration in the resulting biodiesel (FAME) product is below 1% (wt/wt).

These and still other objectives and advantages of the present invention will be apparent from the description which follows. In the detailed description below, preferred embodiments of the invention will be described in reference to the accompanying drawings. These embodiments do not represent the full scope of the invention. Rather the invention may be employed in other embodiments. Reference should therefore be made to the claims herein for interpreting the breadth of the invention.

The main improvement brought by the invention is partial or complete removal of caustic polishing and the corresponding yield loss, salt side stream, and waste water. It is also a further step towards substitution of the less environmentally friendly and currently market-dominating chemical process.

### BRIEF DESCRIPTION OF DRAWINGS

FIG. 1 shows a flow chart over one embodiment of the process according to the invention.

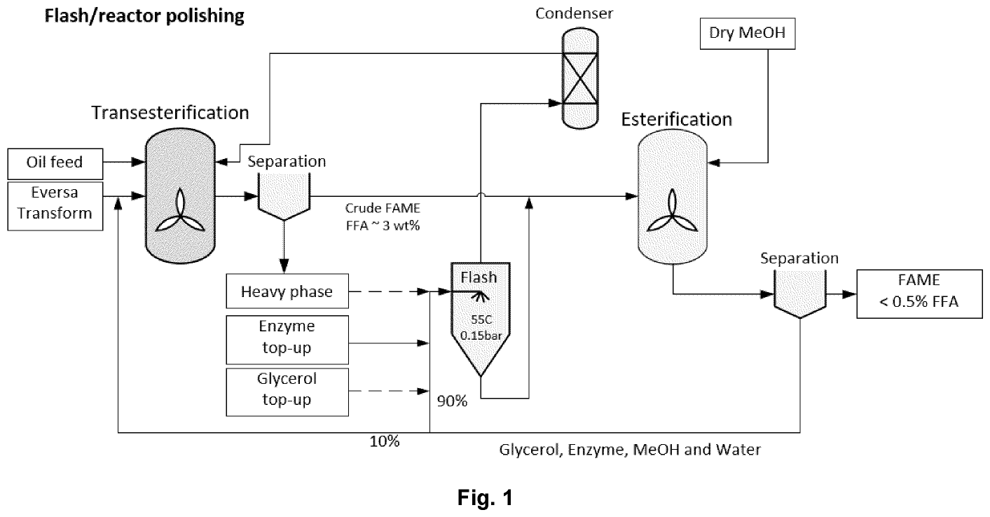

The FIGURE have been included for illustration purposes alone and should not be construed as limiting the invention.

### Definitions

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a step” includes reference to one or more of such steps.

As used herein, “substantial” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition or are included only in amounts which are small enough so as to have no deleterious effect on the composition.

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value and can include a range of plus or minus two standard deviations around the stated value.

Likewise, reference to a gene or polypeptide that is “derived from” another gene or polypeptide X, includes the gene or polypeptide X.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 percent to about 20 percent should be interpreted to include not only the explicitly recited concentration limits of 1 percent to about 20 percent, but also to include individual concentrations such as 2 percent, 3 percent, 4 percent, and sub-ranges such as 5 percent to 15 percent, 10 percent to 20 percent, etc.

Lipid: The term “lipid” refers to phospholipids and their derivatives, triglycerides and derivatives, sterols, stanols, cholesterol, sphingolipids, ceramides, fatty acids, fatty alcohols, glycolipids, proteolipids, lipopolysaccharides, ether-lipids, polar and non-polar lipids and derivatives thereof.

Esterification: The term “esterification” as used herein, refers to a reaction for combining an organic acid such as a fatty acid with any alcohol or polyol such as a glycerol.

Hydrolysis: The term “hydrolysis” as used herein, refers to the reaction of water with an ester to produce an acid and an alcohol.

Alcoholysis: The term “alcoholysis” as used herein, refers to the reaction of an ester with a monohydric alcohol, such as ethanol, butanol, or polyhydric alcohol as glycerol, to produce an ester with a different alkyl group.

Acidolysis: The term “acidolysis” as used herein, refers to the reaction of an ester with an acid leading to the exchange of acyl groups.

Interesterification: The term “interesterification” as used herein, refers to the reaction of a first ester with a second ester leading to a mix up between the acyl and the alcohol moieties.

Transesterification: The term “transesterification” as used herein, refers to any of the following reactions: alcoholysis, acidolysis and interesterification.

Synthesis: The term “synthesis” or “synthesis of fatty acids” as used herein, refer to covalently binding a fatty acid at the sn-2 position of a glyceride, preferably by a one-step reaction selected from any one of the following reactions: esterification, interesterification, alcoholysis, acidolysis, transesterification.

The terms “alkyl” or “alkyl group” is to be construed according to its broadest meaning, to describe a univalent aliphatic compound comprising hydrocarbons.

The terms “glycerol derivatives” and “glycerides” are interchangeably used herein to describe esters, ethers and other derivatives of glycerol in which at least one of the hydrogens, of any of the hydroxyl group attached to the CI, C2 or C3 carbons, is substituted. Examples of glycerol derivatives are: tristearoylglycerol (or tri-Ostearoyl glycerol or glycerol tristearate, or glyceryl tristearate); 1,3-benzylideneglycerol (or 1,3-O-benzylideneglycerol); and glycerol 2-phosphate (or 2-phosphoglycerol) among others. If the substitution is on a carbon atom, rather than on the oxygen of the hydroxyl group than the compound may be considered as a derivative of glycerol (e.g., 1,2,3-nonadecanetriol for C16H33CHOH—CHOH—CH2OH, which may be also considered as 1-C-hexadecyl glycerol). The term “glycerol” as used herein is intended to encompass glycerol derivatives.

Esterase: The terms “Esterase”, “lipase”, “lipase enzyme”, “lipolytic enzyme”, “lipid esterase”, “lipolytic polypeptide”, and “lipolytic protein” means a hydrolase enzyme that splits esters into an acid and an alcohol in a chemical reaction with water call hydrolysis. The term also refers to enzyme referred to as carboxylic ester hydrolyases, referring to enzymes acting on ester bonds, and includes enzymes classified in EC 3.1.1 carboxylic ester hydrolases according to Enzyme Nomenclature (available at http://www.chem.qmw.ac.uk/iubmb/enzyme or from Enzyme Nomenclature 1992, Academic Press, San Diego, California, with Supplement 1 (1993), Supplement 2 (1994), Supplement 3 (1995), Supplement 4 (1997) and Supplement 5, in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650; respectively). Non-limiting examples of esterases include carboxylesterase, arylesterase, triacylglycerol lipase, acetylesterase, acetylcholinesterase, cholinesterase, tropinesterase, pectinesterase, sterol esterase, chlorophyllase, L-arabinonolactonase, gluconolactonase, uronolactonase, tannase, retinyl-palmitate esterase, hydroxybutyrate-dimer hydrolase, acylglycerol lipase, 3-oxoadipate enol-lactonase, 1,4-lactonase, galactolipase, 4-pyridoxolactonase, acylcarnitine hydrolase, aminoacyl-tRNA hydrolase, D-arabinonolactonase, 6-phosphogluconolactonase, phospholipase A1, 6-acetylglucose deacetylase, lipoprotein lipase, dihydrocoumarin lipase, limonin-D-ring-lactonase, steroid-lactonase, triacetate-lactonase, actinomycin lactonase, orsellinate-depside hydrolase, cephalosporin-C deacetylase, chlorogenate hydrolase, alpha-amino-acid esterase, 4-methyloxaloacetate esterase, carboxymethylenebutenolidase, deoxylimonate A-ring-lactonase, 2-acetyl-1-alkylglycerophosphocholine esterase, fusarinine-C ornithinesterase, sinapine esterase, wax-ester hydrolase, phorbol-diester hydrolase, phosphatidylinositol deacylase, sialate O-acetylesterase, acetoxybutynylbithiophene deacetylase, acetylsalicylate deacetylase, methylumbelliferyl-acetate deacetylase, 2-pyrone-4,6-dicarboxylate lactonase, N-acetylgalactosaminoglycan deacetylase, juvenile-hormone esterase, bis(2-ethylhexyl) phthalate esterase, protein-glutamate methylesterase, 11-cis-retinyl-palmitate hydrolase, all-trans-retinyl-palmitate hydrolase, L-rhamnono-1,4-lactonase, 5-(3,4-diacetoxybut-1-ynyl)-2,2′-bithiophene deacetylase, fatty-acyl-ethyl-ester synthase, xylono-1,4-lactonase, N-acetylglucosaminylphosphatidylinositol deacetylase, cetraxate benzylesterase, acetylalkylglycerol acetylhydrolase, and acetylxylan esterase. Non-limiting examples of esterase include carboxylic ester hydrolases classified in EC 3.1.1.1 through and including EC3.1.1.85 according to the Enzyme Nomenclature (available at a website having the address www.chem.qmw.ac.uk/iubmb/enzyme). Esterases have wide specificity; and also may hydrolyze vitamin A esters. Esterases may also come from microsomes that also catalyze the reactions of EC 3.1.1.2, EC 3.1.1.5, EC 3.1.1.6, EC 3.1.1.23, EC 3.1.1.28, EC 3.1.2.2, EC 3.5.1.4, and EC 3.5.1.13. It may have lipase activity (triacylglycerol lipase, EC3.1.1.3), cutinase activity (EC3.1.1.74), sterol esterase activity (EC3.1.1.13) and/or wax-ester hydrolase activity (EC3.1.1.50). For purposes of the present invention lipase activity (i.e. the hydrolytic activity of the lipase) may be determined with a pNP assay using substrates with various chain length.

Parent or parent esterase: The term “parent” or “parent esterase” means an esterase to which an alteration is made to produce the enzyme variants. The parent esterase may be a naturally occurring (wild-type) polypeptide but may also be a variant and/or fragment thereof.

Sequence identity: The relatedness between two amino acid sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment-Total Number of Gaps in Alignment)

Substrates: Suitable substrates for production of fatty acid alkyl esters in accordance with the present invention are a broad variety of vegetable oils and fats; rapeseed and soybean oils are most commonly used, though other crops such as mustard, sunflower, canola, coconut, hemp, palm oil and even algae. The substrate can be of crude quality or further processed (refined, bleached and deodorized). Also, animal fats including tallow, lard, poultry, marine oil as well as waste vegetable and animal fats and oil, commonly known as yellow and brown grease can be used. The suitable fats and oils may be pure triglyceride or a mixture of triglyceride and free fatty acids, commonly seen in waste vegetable oil and animal fats. The substrate may also be obtained from vegetable oil deodorizer distillates. The type of fatty acids in the substrate comprises those naturally occurring as glycerides in vegetable and animal fats and oils. These include oleic acid, linoleic acid, linolenic acid, palmetic acid, steric acid, and lauric acid to name a few. Minor constituents in crude vegetable oils are typically phospholipids, free fatty acids and partial glycerides i.e., mono- and diglycerides. When used herein the phrase “fatty acid residues” refers to fatty acids, either free or esterified as in triglycerides, diglycerides, monoglycerides or fatty acid alkyl esters.

Biodiesel: Fatty acid alkyl esters (FAAE) of short-chain alcohols, such as fatty acid methyl esters (FAME) and fatty acid ethyl esters (FAEE) are also called biodiesel, because they are used as an additive to or as replacement of fossil fuel. Biodiesel constitutes an increasingly important additive or substitute for diesel fuels based on fossil oil because it is produced from renewable resources.

Alcohol: Alcohol used in the process of the invention is preferably a short-chain, branched, or linear alcohol having 1 to 5 carbon atoms (C1, C2, C3, C4, or C5, “a lower alcohol”) or mixtures thereof. Preferred lower alcohols are methanol, ethanol, propanol or mixtures thereof. The alcohol content is preferably less than 4.0, 3.5, 3.0, 2.5, 2.0, 1.5 or 1.0 molar equivalents to the amount of fatty acids in the reaction mixture (free and glyceride bound fatty acids). The alcohol may be added stepwise (such as in 1, 2, 3, 4, 5, 6, 7, 8, 9, 10 or more steps) and/or continuously to the reaction mixture. Addition might be in one, some, or all reactors, if more than one reactor is used in series and/or in parallel, and addition may be uniform with similar dosage at all dosage points or non-uniform with varying dosage rates.

The term “equilibrium” here may be defined as the point where there is no further net reduction of free fatty acids in the reaction mixture, or for example the reduction in FFA is so small that it does not pay off to allow the reaction to continue in the transesterification vessel.

Added glycerol: The term “added glycerol” is glycerol added to the reaction at any point. It can already be present as free glycerol in the feedstock oil. It does not account for the bound glycerol that is freed as byproduct by reaction of glycerides.

Fatty Acid Feedstock: The term “fatty acid feedstock” or “oils and/or fats” or “vegetable oil feedstock” is defined herein as a substrate comprising fatty acid derivatives. The substrate may comprise fatty acid alkyl esters, triglyceride, diglyceride, monoglyceride, free fatty acid or any combination thereof. Any oils and fats of vegetable or animal origin comprising fatty acids may be used as substrate for producing fatty acid alkyl esters in the process of the invention. Also, fatty acid feedstock consisting substantially of fatty acid alkyl esters is suitable as feedstock (biodiesel feedstock) for the present invention. Preferably, the free fatty acid content of the fatty acid feedstock is above 0.25%, above 0.30%, above 0.35%, above 0.50%, above 0.75%, above 1.0%, above 5.0%, above 10.0%, above 15.0%, above 20.0%, above 25.0%, above 30.0%, above 40%, or even above 50.0%. The fatty acid feedstock may be oil selected from the group consisting of: microbial oil, algae oil, canola oil, coconut oil, castor oil, coconut oil (copra oil), corn oil, cottonseed oil, flax oil, fish oil, grape seed oil, hemp oil, jatropha oil, jojoba oil, mustard oil, canola oil, palm oil, distillers' corn oil, palm stearin, palm olein, palm kernel oil, peanut oil, rapeseed oil, rice bran oil, safflower oil, soybean oil, sunflower oil, tall oil, and oil from halophytes, pennycress oil, camelina oil, jojoba oil, coriander seed oil, meadowfoam oil, seashore mallow oil, or any combination thereof.

The fatty acid feedstock may be fat selected from the group consisting of animal fat, including tallow from pigs, beef and sheep, lard, chicken fat, fish oil, or any combination thereof.

The fatty acid feedstock may be crude, refined, bleached, deodorized, degummed, or any combination thereof.

Food quality oils and fats are expensive, and therefore, waste and by-products from their processing as well as non-food grade oils and fats have become increasingly attractive feedstock for producing fatty acid alkyl ester. Soap stock is the fraction of oil obtained in an oil refinery by treating the oil with a base to convert free fatty acids to soaps (e.g., sodium soaps). The soap stock usually contains a fraction of glycerides beside the soaps. Acid oil is the by-product from the oil refinery produced by acidification of soap stock to solubilize the soaps. It mainly contains free fatty acids (FFA) and acylglycerols. Distillates like Palm Fatty Acid Distillate (PFAD) is the by-product from oil refining coming from a distillation process used to eliminate free fatty acid from the oil.

The term fatty acid feedstock is used herein interchangeably with the term biodiesel feedstock.

The fatty acid feedstock is a crude, refined or spent/waste oil or mixtures thereof. The feedstock may be an intermediate product, a waste product or a by-product of oil or fat refining selected from the group consisting of: soap stock; acid oil; fatty acid distillates such as PFAD, soy fatty acid distillate, rapeseed fatty acid distillate, rice bran fatty acid distillate, poultry fat fatty acid distillate, beef tallow fatty acid distillate, etc.; gums from degumming; by-products from the production of omega-3 fatty acids derivates from fish oil; fat trap grease; yellow grease, and brown grease, free fatty acids like oleic acid; or fractions of oil obtained by physical separations; or any combinations thereof.

### DETAILED DESCRIPTION OF THE INVENTION

The present invention relates to a process for reducing the level of free fatty acids in biodiesel/fatty acid alkyl esters.

In one aspect of the invention, the process for reducing level of free fatty acids in biodiesel/fatty acid alkyl esters, said process comprising steps of:
(i) providing a fatty acid feedstock substrate comprising triglycerides, diglycerides, monoglycerides, free fatty acids, fatty acid esters, or any combination thereof;
(ii) reacting said fatty acid feedstock substrate with alcohol in the presence of one or more esterases and added glycerol to produce fatty acid alkyl esters.
(iii) separating the reaction mixture of step (ii) into light phase comprising fatty acid methyl ester (FAME) and a heavy phase comprising esterase, glycerol, short chain alcohol and water;
(iv) drying the mixture of step ii) followed by separation of the light and heavy phases; and/or
(v) drying the heavy phase of step (iii) in presence of the esterase, wherein the glycerol and esterase of the heavy phase is recycled and added back to step (ii); and
(vi) wherein the resulting level of free fatty acid (FFA) concentration in the resulting biodiesel (FAME) product is below 1% (wt/wt).

In one aspect of the invention, the fatty acid feedstock substrate comprises triglycerides, diglycerides, monoglycerides, free fatty acids, fatty acid esters, or any combination thereof.

In one aspect of the invention, the fatty acid feedstock substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, used cooking oil, palm oil mill effluent and brown grease or any combination thereof.

Some fatty acid feedstock substrates have high levels of free fatty acids. For example, fatty acid distillates from palm or soy, acid oils, animal fats or similar feedstocks that can contain from 10-90% FFA. Fatty acid feedstock substrate may be crude biodiesel from another enzymatically catalyzed transesterification reaction with residual FFA remains unconverted. Such crude biodiesel may hold FFA in the range 0.25-8 wt % such as 0.5-6 wt % or even 0.6-5 wt % FFA, the rest being FAME and 0.25-5 wt % unconverted mono-, di-, and triglycerides. Such substrate may hold a significant amount of mono-, di- and triglycerides with varying amounts of FAME and FFA.

In another aspect of the invention, the substrate is distilled crude FAME from light phase separated from heavy phase holding primarily FAME and FFA with traces of glycerides. In such a case the FFA concentration is largely the same as originally present in the crude FAME, but the feedstock quality is markedly improved yielding a better process in terms of rate of reaction and product quality. Such distillation might be conducted at temperatures ranging from 140° C. to 240° C. at a vacuum of 0.5-100 mbara.

In another aspect of the invention, the fatty acid feedstock substrate comprises fatty acids in an amount in the range of from 0.25 to 10 wt %, such as 1 to 8 wt %, 1 to 7 wt %, 1 to 5 wt %, or 1 to 4 wt %.

In one aspect of the invention, the fatty acid feedstock substrate is reacted with alcohol in the presence of one or more esterases.

In one aspect of the invention, the fatty acid feedstock substrate is reacted with alcohol in the presence of one or more esterases and glycerol to produce fatty acid alkyl esters.

In another aspect of the invention, the process is used to convert FFA levels, while leaving significant concentrations of unconverted glycerides.

In one aspect of the invention, the process is a single enzymatic transformation of fatty acid feedstock substrate to biodiesel.

The process is divided into several processing steps with any unit operation in between. Such intermediate unit operations might be, but are not limited to, transportations (e.g. to other parts of the plant or other plants or storage facilities), filtrations, distillation, bleaching, washing, storage. Such separation of the process into two or more parts might be beneficial when an initial first reaction step yielding partial conversion is possible at an existing part of a plant without full reaction being possible e.g. due to equipment constraints. In such a case it could be beneficial to utilize the capacity of such a plant before transporting the partially converted oil to other parts of the same or other plant(s) where final conversion is achievable. Another beneficial reason to separate the process into parts with unit operations in between may improve efficiency of removal of pre-existing impurities after partial conversion to alkyl esters. Such is the case, when using a waste-oil with much metal such as POME—in that case, it is beneficial first to produce low-boiling alkyl esters while reducing high-boiling glycerides before distilling off the partially (or fully) converted alkyl-esters and FFA while the distillation bottoms contain the impurities.

In another aspect of the invention, the process may be viewed as a two-step enzymatic transformation of fatty acid feedstock substrate to biodiesel.

In another aspect of the invention, the process comprises steps of:
(i) providing a fatty acid feedstock substrate comprising triglycerides, diglycerides, monoglycerides, free fatty acids, fatty acid esters, or any combination thereof;
(ii) reacting said fatty acid feedstock substrate with alcohol in the presence of one or more esterases and added glycerol to produce fatty acid alkyl esters;
(iii) separating the reaction mixture of step (ii) into light phase comprising fatty acid methyl ester and a heavy phase comprising esterase, glycerol, short chain alcohol and water;
(iv) drying the heavy phase of step (iii) in presence of the esterase, wherein the glycerol and esterase of the heavy phase is recycled and added back to step (ii); and wherein the resulting level of free fatty acid (FFA) concentration is below 1% (wt/wt).

In the said process, the first step is enzymatic transesterification by esterase, and the second step is the esterification of free fatty acids to biodiesel by esterase. Esterase used in transesterification and esterification may be same esterase or another esterase. In this case, the substrate in step (i) could be the light phase of the reaction mixture from enzymatic transesterification.

Alternatively, the substrate in step (i) could be the full reaction mixture from transesterification reaction which is then dried to drive the esterification process. It could also be the full reaction mixture from transesterification where the heavy phase is isolated by separation and dried either continuously during reaction at any point start and endpoint or in a stepwise fashion before being recirculated into the transesterification reaction.

The mixture of step (ii) is then incubated in an enzyme reactor. The enzyme reactor may be any vessel suitable for enzyme incubation, and the parameters for the incubation are selected in order to facilitate the enzyme reaction. A person of skill in the art is aware of the parameters, and will be able to select e.g., the temperature, mixing, vessel to support this enzyme reaction.

In one aspect of the invention, the enzyme reaction mixture i.e., the mixture comprising enzyme reaction products and remains of the substrate provided and mixed together in step (ii), is subjected to drying. Such drying might be continuous during reaction, e.g. with vacuum applied directly to the reaction vessel while continuously adding methanol to substitute what evaporates. Such drying might also be done in an external vessel looping the reaction mixture and feeding the dried mixture back to the reaction vessel. Drying might also be done by stripping using a suitable gas. Such drying might also be stepwise and/or conducted stepwise between sequential reaction vessels.

In another aspect of the invention, step iv is used to dry the reaction mixture, followed by separation of the light and heavy phases. The dry heavy phase is then recirculated back to step ii.

In one aspect, the reacted mixture of step (ii) is separated. Separation of the reacted mixture is typically done using conventional methods known in the industry.

The enzyme reaction mixture may be subjected to gravitation-based separations such as centrifugation and/or decantation, which results in a light phase and a heavy phase. The light phase contains the FAME, i.e., biodiesel and residual FFA and glycerides. This may optionally be dried in a drying step, i.e., a conventional drying at temperature and conditions typically used to dry biodiesel. It may also optionally be distilled, which is preferred in cases where the feedstock quality does not allow for biodiesel according to lawful specifications. The heavy phase comprises glycerol, esterase, water, and short chain alcohol and will go through the step v.

In one aspect of the present invention, water is added in step ii) is less than 2% wt/wt of the fatty acid feedstock.

In one aspect of the present invention, added glycerol added in step ii) is at least 2% wt/wt of the fatty acid feedstock.

In one aspect of the present invention, the process is continuous and/or batch and/or fed-batch.

The skilled expert in the field will realize that the invention is suitable not only as a standalone or an add-on process with dedicated equipment but might be conducted in existing equipment as an added treatment either in a stepwise treatment following the main transesterification reaction or as a treatment conducted in parallel with the transesterification reaction. The skilled expert in the field would also realize that such optimum designs and operation would depend on whether the process is batch or continuous and would design accordingly. In such cases the amount of added equipment will be reduced because the drying framework will be the main addition.

In one aspect the present invention may benefit where the enzymatic reaction mixture will dry sufficiently in a dryer even at mild conditions allowing for partial enzyme activity. The drying of step (ii) is performed under these mild conditions.

Due to the mild drying conditions that were used in step ii, the esterase still will retain activity, while water and short chain alcohol will be reduced significantly. The water reduction will be sufficient to facilitate esterification to a lower FFA level in the biodiesel when recycled to step ii.

Thus, the heavy phase may be recycled, in its entirety or in part, in the process. The heavy phase may be fed back to the step (ii) in the process, where glycerol, esterase, short chain alcohol is added to the substrate and mixed. In some embodiments, the recycled heavy phase may be supplemented by addition of fresh glycerol, short chain alcohol and/or esterase.

In another aspect of the invention, the fatty acid feedstock optionally also includes a heavy phase comprising primarily glycerol, water and alcohol. Optionally this heavy phase might also comprise enzyme from previous reaction steps and recirculated enzyme from downstream processing steps.

In another aspect of the invention, the substrate comprises free fatty acids in an amount in the range of more than 10 wt %; such as in the range from 10-90 wt %, such as in the range from 10 to 85 wt %, 10 to 80 wt %, 10 to 75 wt %, 10 to 70 wt %, 10 to 65 wt %, 10 to 60 wt %, 10 to 55 wt %, 10 to 50 wt %, 10 to 45 wt %, 10 to 40 wt %, 10 to 35 wt %, 10 to 30 wt %, 10 to 25 wt %, 10 to 20 wt %, 10 to 15 wt % of free fatty acids; or for example 10 to 85 wt %, 15 to 80 wt %, 20 to 75 wt %, 20 to 65 wt %, 20 to 55 wt % of free fatty acids.

In one aspect of the invention, alcohol is one or more of C1-C5 alcohol, preferably ethanol, propanol, methanol or mixtures thereof.

In another aspect of the invention, one or more larger alcohols of <500 g/mol are used. Such as myristyl alcohol having a molar mass of 214.4 g/mol.

In one aspect of the invention, alcohol is dosed so as to achieve a constant concentration of alcohol in the heavy phase of step ii) of 1-50 wt %, such as 2-40 wt % such as 3-30 wt %. The target concentration of alcohol in the heavy phase will depend on enzyme stability, temperature and target product composition.

In another aspect of the invention the alcohol is dosed either stepwise or continuously without regards to the amount of alcohol in the heavy phase. In case several reactors are employed in series or in parallel, the alcohol might be dosed in varying fractions in one, some, or all reactors.

In another aspect of the invention, the total amount of alcohol dosed depends on the feedstock composition and target quality. For example, in case of a near-pure triglyceride oil, a molar excess up to 3, and preferably not more than 2.5 based on convertible fatty acids would be used. While for conversion of e.g. 4.4% FFA without regards to conversion of the glycerides, FFA below 0.25% can be achieved using just 0.2 molar equivalents based on total fatty acids in the feedstock oil.

Drying is a conventional technique for drying biodiesel based on the discrepancy of boiling points for the heavy and light components of the biodiesel reaction. In this invention we are using similar equipment for drying of the heavy phase isolated from the light phase.

It can be difficult to ascertain the temperature inside the drying chamber, and therefore the temperature may be measured at inlet point.

In one aspect of the invention, drying of step iv is done under conditions such that the esterase in step (ii) which has been dried retained at least 40%, preferably 60% and most preferably 80% of its activity.

In another aspect of the invention, the drying step is optionally conducted at optimum drying conditions without regard to enzyme stability and where denatured and inactive enzyme is recycled with the dry glycerol.

In another aspect of the invention, the fact that enzyme tends to form an emulsion layer between the light and heavy phase is utilized. Enzyme-rich emulsion layer might be separated from the light and heavy phase and reused with or without drying treatment. Relative to the case of recycling e.g. ⅔ of the heavy phase and thereby enzyme while purging the remaining ⅓, this principle allows for recycling of >⅔ of the enzyme activity, while <⅓ enzyme is lost with the purged ⅓ of heavy phase. This also possibly allows for efficient, high-temperature drying of the ⅔ recycled glycerol phase, because much enzyme remains intact by bypassing the rough drying conditions.

In one aspect of the invention, glycerol is dried, recirculated and accumulated until the heavy phases comprises 2-40% of the reactor volume prior to any release of bound glycerol from glycerides such as 5-40% preferably 10-40% of the reactor volume.

In one aspect of the invention, the whole—or part of the heavy phase is collected and treated as a whole until a certain dryness is achieved, whereafter it—or part of it is recirculated into step ii.

In another aspect of the invention, a continuous stream of glycerol phase is dried continuously. Part of the added glycerol might be dried continuously while another part is dried non-continuously such as in a batch drying unit.

Water and methanol concentrations in the heavy phase will depend on the accumulation of dried glycerol heavy phase due to dilution. FFA in the light phase at equilibrium is primarily dependent on the ratio of water and methanol in the glycerol phase, and the water concentration and its activity are markedly reduced as a result of glycerol accumulation and this is a main driver of the FFA reduction chemically.

In another aspect of the invention, part of the heavy phase of step iii is purged either prior to and/or after drying, and where the purged fraction is optionally reused in early sequential reaction steps of step ii or further upstream reactions such as the transesterification reactions yielding the optional crude biodiesel feedstock.

In another aspect of the invention, step ii glycerol is added to the process from outside the existing process.

The glycerol might come in varying qualities as long as any pollutants do not significantly inhibit the enzyme.

In another aspect of the invention, glycerol from step ii comes from the transesterification step where the optional crude biodiesel feedstock is produced. Optionally such glycerol is dried and/or refined prior to entering the process.

Methanol-tolerance refers to the decrease in stability (can be measured as thermostability by e.g. DSC) that most esterases show in presence of methanol.

In another aspect of the invention, optionally a single or combination of esterases used in step ii does not include the same enzyme as is used in preparation of the crude biodiesel fatty acid feedstocks in the preceding process steps. Such new esterase is preferably more thermostable and/or methanol-tolerant and/or active at lower water activity levels than the enzyme used for production of the crude biodiesel.

In another aspect of the invention, additional (combination of) esterase(s) is added to the mixture at any point of step ii on top of—or after separation of any preexisting enzymes of previous reaction steps. Such additional esterase(s) is preferably more thermostable and/or methanol-tolerant and/or active at lower water activity levels than the enzyme used for production of the crude biodiesel.

In one aspect of the invention, the esterase(s) is preferably a liquid, granule, dried and/or powder formulation. Less preferably, the enzyme can be employed as immobilized form.

The process according to any of the preceding claims which runs at temperatures of 20-90 C, such as 25-85 C, preferably 30-80 C.

In one aspect of the invention, the process is proceeding in a batch or continuous mode.

In one aspect of the invention, the total duration of the process is from 1-72 hours, such as 2-48 hours, such as 4-40 hours in a batch process.

In one aspect of the invention, wherein the total duration of the process is from 1-72 hours, such as 2-48 hours, such as 4-40 hours in a continuous process.

In another aspect of the invention, the reaction is conducted in e.g. a storage vessel with an initial water and glycerol addition and controlled methanol addition chosen to yield any desired biodiesel quality at equilibrium and corresponding long reaction time. In this case reaction is conducted without regards to reaction time and savings are made on enzyme addition as well as glycerol drying through utilization of slow methanol dosage and reaction time until biodiesel quality is reached. This is e.g. useful and economically beneficial in cases where an idle and ATEX approved (storage) vessel would otherwise stand idle for a significant amount of time.

In one aspect of the invention, the process comprises one or more reactors which are used in series or in parallel.

In another aspect of the invention, the process comprises more than one reactor and where the heavy phases of each or some reactors are separated and dried separately before entering the next reactor.

In another aspect of the invention, more than one reactor is used, and where the heavy phase is separated and dried only between selected reactors such as only between reactor 1 and 2 and 2 and 3 and such as reactor “n” and “n+1”.

Optionally part or all of the heavy phase of reactor “n” might be separated off, optionally dried and optionally recycled back to any previous reactor such as from reactor 4 back to reactor 1 or from 3 back to 1 or 8 back to 5. Optionally the heavy phase of reactor n might be split with only part of the heavy phase being dried before entering different reactors such as separating the contents of reactor 4 into two parts x and y, drying part x and adding it to reactor 3, while adding the undried part y to reactor 1. The combinations are many, and the most preferable combination would result in the most dry composition in the final reactor of any number of sequential and parallel reactors allowing for the lowest FFA at equilibrium in the end product while staying economically attractive.

In another aspect of the invention, optionally part of the light phase of step (iii) is recycled directly back into step (ii). Optionally part of the FAME phase of any reactor n is recycled into previous reactor such as from reactor 4 to reactor 2. Optionally the FAME phase is dried with or without glycerol present during drying.

In one aspect of the invention, the total amount of said esterase enzyme is within the range of 0.01-8 g enzyme protein (EP)/kg of substrate.

In one aspect of the invention, the amount of FFA is reduced to—or kept below 5 wt %, preferably less than 2 wt %, more preferable less than 1 wt % such as 0.5 wt % and most preferably to less than 0.25 wt %.

In one aspect of the invention, when using e.g. a refined oil holding initial FFA below 1% or even close to zero, the invention does not result in an increase in FFA beyond the FFA levels claimed by the invention.

In one aspect of the invention, drying is conducted using drying which is done at a pressure below 250 mbara such as below 150 mbara, preferably below 100 mbara.

In one aspect of the invention, wherein the dried heavy phase mixture of step (iii), obtained in step iv or v has a water content in the range from 0-10 wt %, such as 0.05-8 wt % preferably 0.1-5 wt % and most preferably 0.1-4 wt %.

### Esterase

The esterase provided in step (ii) is one or more esterase, such as one or more enzymes classified as EC 3.1.1 Carboxylic ester hydrolases according to Enzyme Nomenclature (Recommendations of the Nomenclature Committee of the International Union of Biochemistry and Molecular Biology, 1992 or later).

In one aspect, the esterase catalyses the transesterification reaction.

In another aspect, the esterase catalyses the esterification reaction.

In one aspect, the process of the present disclosure includes one or more esterase. Non-limiting examples of suitable esterase include esterase having an amino acid with at least 70%, 80%, 90%, 95%, 96%, 97%, 98%, 99% sequence identity to the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 with esterase activity.

In an aspect of the present disclosure, the esterase comprises an amino acid sequence that has a degree of sequence identity to the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 of at least 60%, e.g., at least 65%, at least 70%, at least 75%, at least 80%, at least 85%, at least 90%, at least 91%, at least 92%, at least 93%, at least 94%, or at least 95%, at least 96%, at least 97%, at least 98%, at least 99%, or at least 100% having esterase activity. In embodiments, suitable esterase in accordance with the present disclosure include the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 having esterase activity. In embodiments, suitable esterase in accordance with the present disclosure include 2 or more of the polypeptides of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 in combination.

In an aspect, the esterase of the present disclosure is an artificial variant comprising a substitution, deletion, and/or insertion of one or more (or several) amino acids of the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10, or a homologous sequence thereof.

In one aspect, one or more esterases may be selected from the group consisting of C. antarctica lipase A as disclosed in WO 88/02775, Thermomyces lanuginosus lipase, Thermomyces lanuginosus (previously Humicola lanuginosus) lipase variants exemplified in WO 00/60063, Humicola insolens cutinase variants disclosed in Example 2 of WO 01/92502, lipases from Humicola lanuginosus (EP 258 068), Chromobacterium Viscosum, Candida rugosa, Pseudomonas cepacia, Geotricum candidum, Rhizomucor miehei, Crytococcus spp. S-2, Candida parapsilosis, Eversa Transform (Novozymes A/S), LIPOZYME CALB L, NS88007 and Callera Trans (Novozymes A/S).

Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a parent polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for cellulolytic enhancing activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identities of essential amino acids can also be inferred from analysis of identities with polypeptides that are related to the parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

In embodiments, the total number of amino acid substitutions, deletions and/or insertions of the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10 is not more than 10, e.g., 1, 2, 3, 4, 5, 6, 7, 8, 9 or 10.

In embodiments, suitable esterase in accordance with the present disclosure comprises, or consists of SEQ ID NOs: 1, 2, 3, 4, 5, 6, 7, 8, 9, or 10 having esterase activity.

One or more (several) components of the esterase in accordance with the present disclosure may be wild-type proteins, recombinant proteins, or a combination of wild-type proteins and recombinant proteins. For example, one or more (several) components may be native proteins of a cell, which is used as a host cell to express recombinantly one or more (several) other components of the esterase composition. One or more (several) components of the esterase composition may be produced as monocomponents, which are then combined to form the enzyme composition. The enzyme composition may be a combination of multicomponent and monocomponent protein preparations.

The esterases used in the processes of the present disclosure may be in any form suitable for use, such as, for example, a crude fermentation broth with or without cells removed, a cell lysate with or without cellular debris, a semi-purified or purified enzyme preparation, or a host cell as a source of the esterases. The esterase composition may be a dry powder or granulate, a non-dusting granulate, a liquid, a stabilized liquid, or a stabilized protected enzyme. Liquid esterase preparations may, for instance, be stabilized by adding stabilizers such as a sugar, a sugar alcohol or another polyol, and/or lactic acid or another organic acid according to established processes.

The esterase can be derived or obtained from any suitable origin, including, bacterial, fungal, yeast, plant, or mammalian origin. The term “obtained” means herein that the esterase may have been isolated from an organism that naturally produces the esterase as a native enzyme. The term “obtained” also means herein that the enzyme may have been produced recombinantly in a host organism employing methods described herein, wherein the recombinantly produced esterase is either native or foreign to the host organism or has a modified amino acid sequence, e.g., having one or more (several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

### Esterase Formulation

The term “esterase” is used herein to refer to the enzyme which catalyzes esterification of FFA to fatty acid methyl esters.

The esterase may be provided in any suitable formulation, such as lyophilized powder, immobilized or in aqueous/liquid solution.

In one embodiment, the esterase is provided as a dry formulation, such as e.g. spray dried, or granulated. If the esterase is provided as a dry formulation, the esterase will be dissolved/suspended in aqueous/liquid solution.

In another embodiment the esterase is provided in a liquid formulation. That is, the esterase is not immobilized on a carrier, but is present in an aqueous or glycerol and/or sorbitol formulation. This represents a cost-saving as liquid formulation enzymes are simpler to produce and therefore cheaper.

Immobilized enzymes, such as Novozym 435, are bound to solid particles, facilitating recovery from reaction mixture and enabling re-use in process. However, the recovery and re-use of immobilized enzyme necessitates additional process steps such filtration. Additionally, small particles might cause fouling or damage to instrumentation lowering the profitability of the process.

Furthermore, the immobilized enzymes themselves are costlier due to the immobilization.

The present invention presents a surprising process for re-use of liquid formulation enzymes, leading to reducing the cost/increasing the process efficiency of use of liquid formulation enzymes.

While the use of esterase in liquid formulation presents advantages, nonetheless if desired, immobilized esterase may be employed.

One particular embodiment relates to the process according to the invention wherein the esterase comprises or consists of CALB in liquid formulation.

Providing the esterase as a liquid has the disadvantage of introducing water into the system (which would shift the equilibrium away from biodiesel production).

However, the inventors have surprisingly found that glycerol caused a significant shift of equilibrium towards an increased conversion of FFA, mitigating the effect of water introduction.

Additionally, the enzyme is recoverable through isolation of the heavy glycerol phase, in which the enzyme resides.

### Advantages

The present invention provides at least the following advantages:
The drying process enables enzyme reaction to proceed further towards the production of fame, increasing yield of FAME.
It is surprising that water can be fully substituted by glycerol when using otherwise water-dependent enzymes. This allows for use of faster and better enzymes than CALB enzyme, which is the only previously described enzyme on this matter. CALB cannot efficiently convert triglycerides and thus it cannot be used in transesterifications, but this invention allows for efficient use of other enzymes, which are capable of transesterifying triglycerides to biodiesel.
Process economy benefits from re-use of the reaction mixture, including re-use of the enzyme and recycling of glycerol.
This improved economy in turn leads to FFA feedstock not previously able to be used can now come into consideration.
Drying total mixture which will save energy costs.
Much smaller drying unit operation as it only processes the heavy phase (glycerol phase) instead of the total reaction mixture.
Optionally not drying the FAME when using step v, is another improvement on opex cost/capital cost due to less material going through heat, vacuum, tank size.

A further benefit is a simple process layout based on conventional engineering unit operations that can be used with lower cost liquid lipase formulation.

The invention is further described in the following paragraphs.

Paragraph 1. A process for reducing level of free fatty acids in biodiesel/fatty acid alkyl esters, said process comprising steps of:
(i) providing a fatty acid feedstock substrate comprising triglycerides, diglycerides, monoglycerides, free fatty acids, fatty acid esters, or any combination thereof;
(ii) reacting said fatty acid feedstock substrate with alcohol in the presence of one or more esterases and added glycerol to produce fatty acid alkyl esters;
(iii) separating the reaction mixture of step (ii) into light phase comprising fatty acid methyl ester (FAME) and a heavy phase comprising esterase, glycerol, short chain alcohol and water;
(iv) drying the mixture of step ii) followed by separation of the light and heavy phases; and/or
(v) drying the heavy phase of step (iii) in presence of the esterase, wherein the glycerol and esterase of the heavy phase is recycled and added back to step (ii); and
   wherein the resulting level of free fatty acid (FFA) concentration in the resulting biodiesel (FAME) product is below 1% (wt/wt).

Paragraph 2. The process according to paragraph 1, wherein said esterase in step ii) is not immobilized.

Paragraph 3. The process according to paragraph 1, wherein said esterase in step ii) is added as a liquid, granule, and/or powder.

Paragraph 4. The process according to anyone of the preceding paragraphs, wherein water added in step ii) is less than 2% wt/wt of the fatty acid feedstock.

Paragraph 5. The process according to anyone of the preceding paragraphs, wherein said added glycerol in step ii) is at least 2% wt/wt of the fatty acid feedstock.

Paragraph 6. The process according to anyone of the preceding paragraphs, wherein the esterase wherein the esterase is capable of transesterification of monoglycerides (MG), diglycerides (Dg), and triglycerides (TG).

Paragraph 7. The process of paragraph 6, wherein the esterase is an esterase of SEQ ID NO: 1, or and esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1.

Paragraph 8. The process according to paragraph 1, wherein said process comprising steps of:
(v) providing a fatty acid feedstock substrate comprising triglycerides, diglycerides, monoglycerides, free fatty acids, fatty acid esters, or any combination thereof;
(vi) reacting said fatty acid feedstock substrate with alcohol in the presence of one or more esterases and added glycerol to produce fatty acid alkyl esters.
(vii) separating the reaction mixture of step (ii) into light phase comprising fatty acid methyl ester and a heavy phase comprising esterase, glycerol, short chain alcohol and water;
(viii) drying the heavy phase of step (iii) in presence of the esterase, wherein the glycerol and esterase of the heavy phase is recycled and added back to step (ii); and wherein the resulting level of free fatty acid (FFA) concentration is below 1 (wt/wt) %.

Paragraph 9. The process according to paragraph 1, wherein water added in step ii) is 2% wt/wt or more of the fatty acid feedstock, and wherein the biodiesel product of claim 1 is reacted with a further esterase in a second esterification step resulting in a FFA concentration below 1%, below 0.5%, such as below 0.3%.

Paragraph 10. The process according to paragraphs 9, wherein the further esterase is an esterase of SEQ ID NO: 3, or and esterase having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 3.

Paragraph 11. The process according to anyone of the preceding paragraphs, wherein the process is continuous or batch/fed-batch.

Paragraph 12. The process according to anyone of the preceding paragraphs, wherein said drying of step v) is conducted to a point where the FFA concentration is below <1 wt %.

Paragraph 13. The process according to anyone of the preceding paragraphs, wherein said alcohol is a C1-C5 alcohol.

Paragraph 14. The process according to anyone of the preceding paragraphs, wherein said fatty acid alkyl esters are methyl- or ethyl-esters.

Paragraph 15. The process according to anyone of the preceding paragraphs, wherein said added glycerol is optionally dried before being recirculated to comprise less than 20 wt % water, preferably less than 10 wt % water and most preferably less than 2 wt % water.

Paragraph 16. The process according to anyone of the preceding paragraphs, wherein the drying of step iv) and/or step v) is done under conditions such that the esterase in step ii) which has been dried, retain at least 40%, preferably 60% and most preferably 80% of its activity.

Paragraph 17. The process according to any of the preceding paragraphs, wherein the drying is conducted in the range from 30° C.-100° C., such as from 40° C. to 90° C. preferably from 45° C. to 85° C. and most preferably from 50° C. to 80° C.

Paragraph 18. The process according to anyone of the preceding paragraphs, wherein the reaction of step ii) is performed at temperatures of 20-90° C., such as 25-85° C., preferably 30-80° C.

Paragraph 19. The process according to anyone of the preceding paragraphs, wherein the substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, used cooking oil, palm oil mill effluent and brown grease or any combination thereof.

Paragraph 20. The process according to anyone of the preceding paragraphs, wherein the total duration of the process is from 1-72 hours, such as 2-48 hours, such as 4-40 hours in a batch process and wherein the total duration of the process is from 1-72 hours, such as 2-48 hours, such as 4-40 hours in a continuous process.

Paragraph 21. The process according to anyone of the preceding paragraphs, wherein the total amount of said esterase is within the range of 5-8000 ppm (wt enzyme protein/wt of substrate).

Paragraph 22. The process according to anyone of the preceding paragraphs, wherein the total amount of said non-immobilized esterase is within the range of 5-1000 ppm (wt enzyme protein/wt of substrate).

Paragraph 23. The process according to anyone of the preceding paragraphs, wherein the amount of FFA is reduced to less than 5 wt %, preferably less than 2 wt %, more preferable less than 1 wt % such as 0.5 wt % and most preferably to less than 0.25 wt %.

Paragraph 24. The process according to anyone of the preceding paragraphs, wherein said esterase is selected from the group consisting of: Aspergillus lipase; Aspergillus niger lipase; Thermomyces lanuginosa lipase; Candida Antarctica lipase A; Candida Antarctica lipase B; Candida cylindracae lipase; Candida deformans lipase; Candida lipolytica lipase; Candida parapsilosis lipase; Mucor miehei, Chromobacterium; Candida rugosa lipase; Corynebacterium acnes lipase; Humicola lanuginose, Cryptococcus spp. S-2 lipase; Fusarium culmorum lipase; Fusarium heterosporum lipase; Fusarium oxysporum lipase; Mucor javanicus lipase; Rhizomucor miehei lipase; Rhizomucor delemar lipase; Burkholderia (Pseudomonas) cepacia lipase; Pseudomonas sp, ATCC 21808, Pseudomonas camembertii lipase; Pseudomonas fluorescens lipase; Rhizopus lipase; Rhizopus arrhizus lipase; Staphylococcus aureus lipase; Geotrichium candidum lipase; Hyphozyma sp. lipase; Klebsiella oxytoca lipase; and wildtype orthologs and homologs thereof; and variants thereof.

Paragraph 25. The process according to anyone of the preceding paragraphs further comprises separation of said fatty acid alkyl esters.

Paragraph 26. The process according to anyone of the preceding paragraphs, wherein the biodiesel product comprises a bound glycerol concentration below 1 wt %, preferably below 0.5 wt % and most preferably below 0.3 wt %.

Paragraph 27. The process according to paragraph 1, wherein the light phase comprises monoglyceride concentration below 0.8 wt %.

Paragraph 28. The process according to paragraph 1, wherein the light phase comprises bound glycerol value below 0.24 wt %.

Paragraph 29. The process according to paragraph 1, wherein the light phase comprises FFA concentration below 0.5 wt %, preferably 0.25 wt %.

Paragraph 30. The process according to paragraph 1, wherein the light phase is further subjected to caustic washing treatment.

Paragraph 31. The process according to paragraph 1 or 30, wherein the light phase is subjected additional treatments such as distillation, washing, drying, bleaching or combination thereof.

Paragraph 32. The process according to any of the preceding paragraphs wherein the process is performed in any number of sequential and/or parallel reactors, wherein the reaction can be partial or full in each.

Paragraph 33. The process according to anyone of the preceding paragraphs, wherein said alcohol is added stepwise and/or continuously, wherein stepwise addition of the alcohol may be in 1, 2, 3, 4, 5, 6, 7, 8, 9, 10 or more steps.

Paragraph 34. The process according to the preceding paragraphs, where more than one reactor is used in series, and where equal or unequal partial amounts of the total amount of alcohol are added in some or all of the reactors.

Paragraph 35. The process according to anyone of the preceding paragraphs further comprises separation of said fatty acid alkyl esters.

Paragraph 36. The process according to paragraph 1, wherein glycerol is recycled partial or fully of the dried reaction mixture of step ii)

Paragraph 37. The process according to paragraph 1, wherein drying the heavy phase of step iii) in presence of an esterase, wherein part or all of the glycerol and esterase of the heavy phase is recycled and added back to step ii) as part or all of said added glycerol.

Paragraph 38. The process according to paragraph 1, wherein one or more esterase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

Paragraph 39. The process according to paragraph 38, wherein one or more esterase comprises or consists of polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

Paragraph 40. The process according to paragraph 13, wherein the alcohol is selected from a group consisting of methanol, ethanol, propanol, butanol or mixtures thereof.

Paragraph 41. The process according to paragraph 40, wherein the alcohol is methanol.

Paragraph 42. The process according to the preceding paragraphs, wherein the FFA is reduced with conversion of triglyceride is less than 30%, preferably less than 20%, and most preferably less than 10%.

Paragraph 43. The process according to previous paragraphs, wherein the crude oil with >1 wt % FFA is reacted to reduce FFA levels below 1 wt %, preferably less than 0.5 wt %, and most preferably below 0.25 wt %.

Paragraph 44. The process according to previous paragraphs, wherein the esterase is selected from a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% sequence identity to the polypeptide of SEQ ID NO: 1.

The present invention is further described by the following examples that should not be construed as limiting the scope of the invention.

### Example 1

Crude palm oil (CPO) comprising 4.4 wt % free fatty acid (FFA), 6.1 wt % diacylglycerol (DAG) was melted at 60° C. and used without prior drying. 30 g CPO was then weighed off in 100 mL square bottle followed by addition of 20% (wt/wt of oil) dry technical grade glycerol. The mixture was conditioned in a shaking incubator oven at 35° C. under 250 rpm mixing for 30 minutes to ensure that the temperature was reached. 100 ppm sodium hydroxide was added as 50% aqueous solution.

Various enzyme dosages of SEQ ID NO: 1 or 2 (liquid and immobilized was added at timepoint 0 hour). Reaction was performed at 35° C. in the shaking incubator at 250 rpm under 40 hours continuous linear dosing of 1.7 molar equivalents of methanol based on free and bound fatty acids. After methanol dosing completion at the 40 hours mark, reaction was continued until the 48 hours mark and the results are measured. 2 mL samples are taken by pipette into 2 mL Eppendorf tubes. Samples are incubated for 10 minutes at 99° C. to inactivate the enzyme before being centrifuged at 2000 rpm for 1 minute. Finally, the samples are dried under vacuum at 80 C for 2 hours to remove methanol.

Method for analysis: FAME, Mono- di and triglycerides by AOCS Official Method Ck 2-09 using the Quality Trait Analysis (QTA) NIR method offered.

TABLE 1

| Enzyme dosage, | | | | |
|---|---|---|---|---|
| % (wt/wt of oil) | MAG | DAG | TAG | FAME |
| 0.2% of SEQ NO: 1 | 0.67 | 0.425 | 0.403 | 95.36 |
| (liquid) | | | | |
| 0.5% of SEQ NO: 1 | 0.4 | 0.275 | 0.092 | 97.16 |
| (liquid) | | | | |
| 0.2% of SEQ NO: 2 | 2.97 | >5.5 | >9.9 | <63 |
| (immobilized) | | | | |
| 0.5% of SEQ NO: 2 | 4.125 | >5.5 | >9.9 | <63 |
| (immobilized) | | | | |
| 1% of SEQ NO: 2 | >10 | >5.5 | >9.9 | — |
| (immobilized) | | | | |
| 2% of SEQ NO: 2 | 7.671 | >5.5 | >9.9 | 74.36 |
| (immobilized) | | | | |
| 5% of SEQ NO: 2 | 0.294 | 0.225 | 0.286 | 97.18 |
| (immobilized) | | | | |
| 10% of SEQ NO: 2 | 0.726 | 0.307 | 0.257 | 95.15 |
| (immobilized) | | | | |

Results show that the immobilized enzyme does not perform at par with the liquid enzyme, requiring a much higher dosage for similar results. It is also worth noting that the enzyme protein concentration of the immobilized formulation is between a factor 1.5 and 2 higher than the liquid formulation. The comparison is between two very similar enzymes, and the large difference in enzyme protein dosages accounts for any difference in efficacy of the molecules. Therefore, it is clear that the liquid enzyme formulation is preferable, while the immobilized enzyme can be dosed to allow for sufficient reaction, however at an unacceptable cost.

### Example 2: Biodiesel Reaction

Crude palm oil (CPO) comprising triglycerides, 4.4 wt % FFA, 6.1 wt % DAG oil was melted at 60° C. 30 g of CPO was then weighed off in 100 mL square bottle before addition of 2% (wt/wt of oil) water. The mixture was conditioned in a shaking incubator oven at 35° C. under 250 rpm mixing for 30 minutes to ensure that temperature was reached. 100 ppm NaOH was added as 50% aqueous solution to adjust the extract-pH reading to around 5.5 (the extract pH-reading is measured by mixing 10 g 0.1 wt % KCL solution with 10 g oil for 30 minutes at 60° C. and measuring pH on the aqueous phase). The NaOH addition was determined from a standard curve previously made on the feedstock oil. 0.2% enzyme of SEQ ID NO: 1 was added at timepoint 0 h. Reaction was performed at 35° C. in the shaking incubator at 250 rpm under 20 h continuous linear dosing of 1.7 molar equivalents of methanol based on free and bound fatty acids. After methanol dosing completion at the 20 hours mark, reaction was continued until the 48 hours mark, after which reaction was considered to have reached equilibrium and the results are measured. 2 mL samples are taken by pipette into 2 mL Eppendorf tubes. Sample was incubated for 10 minutes at 99° C. to inactivate the enzyme before being centrifuged at 2000 rpm for 1 minute. Finally, the samples are dried under vacuum at 80° C. for 2 hours to remove the excess methanol dissolved in the FAME.

FFA by AOCS Official Method Ca 5a-40. Mono- di, and triglycerides by AOCS Official Method Ck 2-09 using the Quality Trait Analysis (QTA) NIR method offered by Eurofins.

TABLE 2
Light phase biodiesel product components

| | Monoglycerides, | Diglycerides, | Triglycerides, | FFA, |
|---|---|---|---|---|
| | wt % | wt % | wt % | wt % |
| 24 h | 1.70 | 1.46 | 0.64 | 1.50 |
| 48 h | 0.41 | 0.16 | 0.04 | 1.32 |

From the above table, remaining sum to reach 100% is largely FAME. Glyceride levels are all within standard specification levels of biodiesel while FFA is markedly above the 0.25 wt % mark. At the end of reaction, there is also a heavy phase which comprises most of the enzyme, the 2% water initially dosed, most of the excess methanol dosed, as well as the 10-13% (wt/wt of oil) glycerol liberated through reaction. Addition of water is standard in today's process and is the reason why FFA levels are elevated relative to the most preferred level of <0.25 wt %.

### Example 3: Glycerol Addition

CPO (4.4 wt % FFA, 6.1 wt % DAG) oil was melted at 60° C. 30 g of CPO was then weighed off in 100 mL square bottle followed by addition of 0.5 or 1% (wt/wt of oil) water and 0.5 or 10% (wt/wt of oil) dry technical grade glycerol. The mixture was conditioned in a shaking incubator oven at 35° C. under 250 rpm mixing for 30 minutes to ensure that the temperature was reached. 100 ppm NaOH is added as 50% aqueous solution. 0.2% (wt/wt of oil) of enzyme of SEQ ID NO: 1 was added at timepoint 0 h. Reaction was performed runs at 35° C. in the shaking incubator at 250 rpm under 20 h continuous linear dosing of 1.7 or 2.0 molar equivalents of methanol based on free and bound fatty acids. After methanol dosing was completed at the 20 h mark, reaction was continued until the 48 hours mark, after which reaction was considered to have reached equilibrium and the results are measured. 2 mL samples are taken by pipette into 2 mL Eppendorf tubes. Samples are incubated for 10 minutes at 99° C. to inactivate the enzyme before being centrifuged at 2000 rpm for 1 minute. Finally, the samples are dried under vacuum at 80° C. for 2 hours to remove methanol.

Methods for analysis: FFA by AOCS Official Method Ca 58-40. Mono- di, and triglycerides by AOCS Official Method Ck 2-09 using the Quality Trait Analysis (QTA) NIR method offered by Eurofins.

TABLE 3

| | | Eq. | Reaction | | | | |
|---|---|---|---|---|---|---|---|
| Glycerol | Water | Methanol/ | Time | MG, | DG, | TG, | FFA, |
| (%) | (%) | 20 h | (hours) | wt % | wt % | wt % | wt % |
| 0 | 0.5 | 1.7 | 24 | 6.11 | 4.73 | 2.04 | — |
| | | | 48 | 5.03 | 3.88 | 1.71 | 1.14 |
| | | 2 | 24 | 7.33 | 5.36 | 2.17 | — |
| | | | 48 | 6.76 | 4.98 | 1.88 | 1.25 |
| | 1 | 1.7 | 24 | 3.16 | 2.53 | 1.16 | — |
| | | | 48 | 2.01 | 1.56 | 0.66 | 1.06 |
| | | 2 | 24 | 4.75 | 3.42 | 1.34 | — |
| | | | 48 | 3.85 | 2.85 | 1.06 | 1.28 |
| 5 | 0.5 | 1.7 | 24 | 1.89 | 1.54 | 0.98 | — |
| | | | 48 | 1.27 | 0.66 | 0.46 | 0.60 |
| | | 2 | 24 | 2.79 | 2.14 | 1.15 | — |
| | | | 48 | 2.08 | 1.44 | 0.68 | 0.68 |
| | 1 | 1.7 | 24 | 1.70 | 1.28 | 0.79 | — |
| | | | 48 | 0.59 | 0.07 | 0.00 | 0.77 |
| | | 2 | 24 | 3.05 | 2.32 | 1.11 | — |
| | | | 48 | 2.21 | 1.60 | 0.60 | 0.85 |
| 10 | 0.5 | 1.7 | 24 | 1.97 | 1.49 | 1.00 | — |
| | | | 48 | 0.94 | 0.41 | 0.28 | 0.48 |
| | | 2 | 24 | 2.92 | 2.15 | 1.21 | — |
| | | | 48 | 2.03 | 1.34 | 0.66 | 0.54 |
| | 1 | 1.7 | 24 | 1.55 | 0.85 | 0.68 | — |
| | | | 48 | 0.45 | 0.00 | 0.00 | 0.63 |
| | | 2 | 24 | 1.77 | 1.22 | 0.66 | — |
| | | | 48 | 1.09 | 0.48 | 0.33 | 0.66 |
| 0 | 2 | 1.7 | 24 | 1.70 | 1.46 | 0.64 | — |
| | | | 48 | 0.41 | 0.16 | 0.04 | 1.32 |

The combination of 0% glycerol, 2% water, 1.7 eq. methanol/20 h dosage, is the standard reaction as described in Example 1. Then, considering first the reaction without added glycerol, it becomes clear that reaction was significantly impeded when less than 2% water was added. The difference in glyceride conversions between 24 and 48 hours was small and suggests stalled reactions unable to reach equilibrium due to enzyme inactivation. Then, considering the results of reactions with initially added glycerol, it is clear that glycerol brings an improvement. Adding 5% glycerol along with just 1% water and 1.7 equivalents of methanol results in a marked improvement relative to the example 1 reaction having acceptable glyceride levels as well as a significantly reduced FFA level after 48 hours of reaction. Adding 10% glycerol brings a further improvement on all parameters, while resulting in near-acceptable glyceride levels with just 0.5% water added. Further, adding 10% (wt/wt of oil) glycerol results in 20-23% glycerol in the total mixture at equilibrium when the glycerides are mostly converted. The marked improvements brought by glycerol are thus clearly demonstrated.

### Example 4: Methanol Dosing

CPO (4.4 wt % FFA, 6.1 wt % DAG) oil was melted at 60° C. 30 g CPO was then weighed off in 100 mL square bottle followed by addition of 0.5 or 1% (wt/wt of oil) water and 0.5 or 10% (wt/wt of oil) dry technical grade glycerol. The mixture was conditioned in a shaking incubator oven at 35° C. under 250 rpm mixing for 30 minutes to ensure that the temperature was reached. 100 ppm NaOH was added as 50% aqueous solution. 0.2% (wt/wt of oil) of enzyme of SEQ ID NO: 1 was added at timepoint 0 hour. Reaction runs at 35° C. in the shaking incubator at 250 rpm under 40 hours continuous linear dosing of 1.7 or 2.0 molar equivalents of methanol based on free and bound fatty acids. After methanol dosing completion at the 40 hours mark, reaction was continued until the 48 hours mark and the results are measured. 2 mL samples are taken by pipette into 2 mL Eppendorf tubes. Samples are incubated for 10 minutes at 99° C. to inactivate the enzyme before being centrifuged at 2000 rpm for 1 minute. Finally, the samples are dried under vacuum at 80° C. for 2 hours to remove methanol.

Methods for analysis: FFA by AOCS Official Method Ca 5a-40. Mono- di, and triglycerides by AOCS Official Method Ck 2-09 using the Quality Trait Analysis (QTA) NIR method offered by Eurofins.

TABLE 4

| | | Eq. | | | | | | |
|---|---|---|---|---|---|---|---|---|
| Glycerol | Water | Methanol/ | | | MG, | DG, | TG, | FFA, |
| (%) | (%) | hour | | Time | wt % | wt % | wt % | wt % |
| 0 | 0.5 | 1.7/40 | h | 48 h | 1.29 | 0.64 | 0.42 | 0.75 |
| | | 2/40 | h | 48 h | 1.52 | 0.75 | 0.45 | 0.78 |
| | 1 | 1.7/40 | h | 48 h | 0.65 | 0.23 | 0.15 | 0.91 |
| | | 2/40 | h | 48 h | 0.78 | 0.30 | 0.17 | 0.89 |
| 5 | 0.5 | 1.7/40 | h | 48 h | 0.58 | 0.11 | 0.04 | 0.60 |
| | | 2/40 | h | 48 h | 0.69 | 0.19 | 0.13 | 0.55 |
| | 1 | 1.7 eq/40 h | | 48 h | 0.58 | 0.00 | 0.00 | 0.75 |
| | | 2/40 | h | 48 h | 0.71 | 0.12 | 0.01 | 0.72 |
| 10 | 0.5 | 1.7/40 | h | 48 h | 0.88 | 0.23 | 0.16 | 0.54 |
| | | 2/40 | h | 48 h | 0.63 | 0.19 | 0.13 | 0.48 |
| | 1 | 1.7/40 | h | 48 h | 0.60 | 0.18 | 0.13 | 0.65 |
| | | 2/40 | h | 48 h | 0.65 | 0.12 | 0.01 | 0.62 |
| 0 | 2 | 1.7/20 | h | 48 h | 0.41 | 0.16 | 0.04 | 1.32 |

Results when compared to the results of Example 2, there is a clear improvement achieved through reduced methanol dosage speed. The above table shows that reducing the amount of added water stresses the enzyme, which was also apparent here, when considering especially the glyceride levels. These results, when compared to example 2, demonstrates that the negative effects of reducing the amount of water added can be mitigated by a gentler dosing regimen of methanol. It is clear, that even though the results of this example are fine in the cases of low water and no glycerol, the conversions are poorer than in the cases where added glycerol accompanies the reduced water dosage.

### Example 5

CPO (4.4 wt % FFA, 6.1 wt % DAG) oil was dried in a rotary evaporator at 80° C. under 5 mbar vacuum for 1 hour. 30 g pre-dried CPO was then weighed off in 100 mL square bottle followed by addition of 20% (wt/wt of oil) dry technical grade glycerol. The mixture was conditioned in a shaking incubator oven at 35° C. under 250 rpm mixing for 30 minutes to ensure that the temperature was reached. 100 ppm NaOH was added as 50% aqueous solution. 0.25 or 0.5% (wt/wt of oil) enzyme of SEQ ID NO: 1 was added at timepoint 0 h. Reaction runs at 35° C. in the shaking incubator at 250 rpm under 40 h continuous linear dosing of 2 molar equivalents of methanol based on free and bound fatty acids. After methanol dosing completion at the 40 hours mark, reaction is continued until the 72 hours mark and the results are measured. 2 mL samples are taken by pipette into 2 mL Eppendorf tubes. Samples are incubated for 10 minutes at 99° C. to inactivate the enzyme before being centrifuged at 2000 rpm for 1 minute. Finally, the samples are dried under vacuum at 80 C for 2 hours to remove methanol.

Methods for analysis: FFA by AOCS Official Method Ca 5a-40. Mono- di, and triglycerides by AOCS Official Method Ck 2-09 using the Quality Trait Analysis (QTA) NIR method offered.

TABLE 5

| Enzyme Dosage | | | | | |
|---|---|---|---|---|---|
| (Lipase of | Reaction | MG, | DG, | TG, | FFA, |
| SEQ No: 1) | Time | wt % | wt % | wt % | wt % |
| 0.25% | 24 h | 3.78 | 3.68 | 5.65 | 0.39 |
| | 48 h | 1.27 | 0.61 | 0.36 | 0.21 |
| | 72 h | 0.7 | 0.18 | 0.06 | 0.24 |
| 0.5% | 24 h | 3.06 | 2.9 | 4.22 | 0.46 |
| | 48 h | 0.67 | 0.11 | 0.05 | 0.23 |
| | 72 h | 0.6 | 0.06 | 0.02 | 0.25 |

Results show EN-14214-compliant glyceride and FFA levels are achieved using the enzyme of SEQ ID NO: 1 in a single reaction step on a standard palm oil quality. Using 0.25% enzyme enables acceptable results in 72 hours while doubling of the enzyme dosage enables similar results in 48 hours.

### Examples 6

30 g crude biodiesel with 4.9 wt % FFA, 1 wt % monoglyceride, 0.3% diglyceride, <0.1 wt % triglyceride. The crude biodiesel is prepared using crude palm oil (CPO) with 0.3% SEQ ID NO: 3 (per weight of CPO) with 2% water added (per weight of CPO) and 1.7 equivalents of methanol relative (to moles of fatty acids in the feedstock) dosed over 20 hours with the final crude FAME product finished after 24 hours. The crude FAME stemming from reaction has then been dried at 5 mbar vacuum at 60° C. for 16 hours before usage in the reaction. The reaction on 30 g crude biodiesel runs with 0.1% CALB solution, 5 or 20% technical grade (>99%) glycerol added and with varying amounts of methanol.

FFA measured using AOCS Official Method Ca 5a-40. MG and BG measured by Eurofins QTA.

TABLE 6

| Glycerol | Methanol | Time | FFA | MG | BG |
|---|---|---|---|---|---|
| 5% | 4% | 2 h | 2.02 | 0.97 | 0.26 |
| | | 24 h | 0.46 | 0.58 | 0.15 |
| | 6% | 2 h | 2.12 | 0.95 | 0.25 |
| | | 24 h | 0.49 | 0.63 | 0.17 |
| | 8% | 2 h | 2.69 | 1.01 | 0.26 |
| | | 24 h | 0.20 | 1.03 | 0.26 |
| | 10% | 2 h | 2.80 | 0.96 | 0.23 |
| | | 24 h | 0.21 | 0.89 | 0.23 |

| | | | |
|---|---|---|---|
| Feedstock | 4.91 | 1.03 | 0.29 |

TABLE 7

| Glycerol | MeOH | Time | FFA | MG | BG |
|---|---|---|---|---|---|
| 20% | 4% | 2 h | 1.88 | 0.80 | 0.23 |
| | | 24 h | 0.23 | 0.64 | 0.17 |
| | 6% | 2 h | 1.79 | 0.84 | 0.24 |
| | | 24 h | 0.20 | 0.66 | 0.17 |
| | 8% | 2 h | 1.61 | 0.86 | 0.24 |
| | | 24 h | 0.18 | 0.73 | 0.19 |
| | 10% | 2 h | 1.90 | 1.00 | 0.27 |
| | | 24 h | 0.18 | 0.61 | 0.16 |

| | | | |
|---|---|---|---|
| Feedstock | 4.91 | 1.03 | 0.29 |

Glycerol has a large effect both on rate and on the impact of methanol on the activity of the enzyme. 2 hours measurements are a good enough representation of the rate, the enzyme is much more active at 4% methanol than at 10% methanol when 5% glycerol is added relative to 8% methanol being optimum with 20% glycerol added. It is furthermore significant that within specification levels of FFA, MG and BG can be achieved in a single lab batch experiment using 20% glycerol. Specification levels at the point of writing this are <0.25 wt % FFA, <0.8 wt % MG and <0.24 wt % BG. BG is bound glycerides, which is a summarized value for the total amount of MG, DG and TG.

### Example 7: FFA Removal Prior to Chemical Biodiesel Production

In chemical biodiesel production using methoxide, FFA cannot be present at concentrations above 0.5 wt % in the feedstock, because it will result in information of soap and degrade the catalyst. Removal of FFA through formation of glycerides or FAME by chemical or uncatalyzed reaction with glycerol has been tried and is currently employed today. Additionally, some producers remove the FFA by a deodorization step conducted at >220 C. However, the method described in this filing has also shown applicability in this treatment and an example is shown here. Rate of reaction is the critical point in this reaction, which is considered a pretreatment industrially, with little operating cost available. Therefore, the main point of this example is to show the quick reduction in FFA that can be achieved in a crude oil when using glycerol to stabilize while immediately dosing an amount of methanol and using largely TAG-inactive CALB enzyme to esterify the FFA while trans-esterifying the DAG.

The feedstock is CPO (4.4 wt % FFA, 6.1 wt % DAG), the rest being mainly TAG. CALB enzyme is employed because it shows little reactivity on TAG, which is beneficial because it allows for dosage of methanol stoichiometrically only to the FFA, MAG and DAG. Methanol is does and reported below in equivalents based on total fatty acids in the feedstock oil. The oil is melted, and 30 g is weighed off. Then glycerol and methanol was added, and the mixture is pre-heated to 40 C. Finally, the enzyme is dosed at time zero. In the tables below, the remainder up to 100% is triglyceride and small amounts of MG, which was not measured here.

Methods for analysis: FFA by AOCS Official Method Ca 5a-40. FAME measured by a customized NMR method. DAG by a customized HPLC method.

TABLE 8

| | | | | | FFA | FAME | DAG |
|---|---|---|---|---|---|---|---|
| | | | | | (wt %) | (wt %) | (wt %) |
| 0.1 equiv | 0% Glycerol | 0.1% SEQ | 2 | h | 1.58 | 4.70 | 4.78 |
| MeOH | | ID NO: 3 | 24 | h | 1.11 | 4.50 | 3.27 |
| | | 0.46% SEQ | 2 | h | 1.68 | 4.82 | 3.34 |
| | | ID NO: 3 | 24 | h | 1.80 | 7.68 | 2.13 |
| | 5% Glycerol | 0.1% SEQ | 2 | h | 1.78 | 3.91 | 3.64 |
| | | ID NO: 3 | 24 | h | 1.51 | 7.64 | 2.51 |
| | | 0.46% SEQ | 2 | h | 0.58 | 6.42 | 2.88 |
| | | ID NO: 3 | 24 | h | 2.81 | 8.09 | 2.74 |
| | | 0.1% SEQ | 2 | h | 0.89 | 5.78 | 2.95 |
| | | ID NO: 3 | 24 | h | 1.45 | 7.93 | 2.33 |
| | | 0.46% SEQ | 2 | h | 0.47 | 7.07 | 1.25 |
| | 10% Glycerol | ID NO: 3 | 24 | h | 2.57 | 7.99 | 2.80 |
| 0.2 equiv | 0% Glycerol | 0.1% SEQ | 2 | h | 3.07 | 1.02 | 5.58 |
| MeOH | | ID NO: 3 | 24 | h | 0.66 | 5.39 | 10.55 |
| | | 0.46% SEQ | 2 | h | 1.12 | 3.58 | 4.07 |
| | | ID NO: 3 | 24 | h | 0.46 | 7.59 | 2.99 |
| | 5% Glycerol | 0.1% SEQ | 2 | h | 0.95 | 5.92 | 2.86 |
| | | ID NO: 3 | 24 | h | 0.52 | 8.27 | 2.61 |
| | | 0.46% SEQ | 2 | h | 0.27 | 7.42 | 2.26 |
| | | ID NO: 3 | 24 | h | 1.34 | 10.16 | 2.26 |
| | | 0.1% SEQ | 2 | h | 0.88 | 6.08 | 2.91 |
| | | ID NO: 3 | 24 | h | 0.51 | 8.52 | 1.74 |
| | | 0.46% SEQ | 2 | h | 0.23 | 7.75 | 2.45 |
| | 10% Glycerol | ID NO: 3 | 24 | h | 1.74 | 12.22 | 2.64 |

TABLE 9

| | | | | | FFA | FAME | DAG |
|---|---|---|---|---|---|---|---|
| | | | | | (wt %) | (wt %) | (wt %) |
| 0.4 equiv | 0% Glycerol | 0.1% SEQ | 2 | h | 3.73 | 0.16 | 5.52 |
| MeOH | | ID NO: 3 | 24 | h | 3.71 | 0.59 | 6.96 |
| | | 0.46% SEQ | 2 | h | 2.85 | 1.64 | 5.14 |
| | | ID NO: 3 | 24 | h | 1.51 | 4.27 | 35.80 |
| | | | | | | | (outlier) |
| | 5% Glycerol | 0.1% SEQ | 2 | h | 2.13 | 3.47 | 4.15 |
| | | ID NO: 3 | 24 | h | 0.20 | 8.47 | 4.56 |
| | | 0.46% SEQ | 2 | h | 0.33 | 7.06 | 3.12 |
| | | ID NO: 3 | 24 | h | 0.42 | 9.17 | 3.50 |
| | 10% Glycerol | 0.1% SEQ | 2 | h | 1.22 | 5.54 | 3.31 |
| | | ID NO: 3 | 24 | h | 0.29 | 9.48 | 3.86 |
| | | 0.46% SEQ | 2 | h | 0.19 | 7.67 | 2.96 |
| | | ID NO: 3 | 24 | h | 0.58 | 11.36 | 3.70 |
| 1 equiv | 0% Glycerol | 0.1% SEQ | 2 | h | 3.62 | 0.62 | 5.71 |
| MeOH | | ID NO: 3 | 24 | h | 3.55 | 0.73 | 6.82 |
| | | 0.46% SEQ | 2 | h | 3.21 | 1.12 | 5.56 |
| | | ID NO: 3 | 24 | h | 2.47 | 2.69 | 6.29 |
| | 5% Glycerol | 0.1% SEQ | 2 | h | 3.72 | 0.56 | 5.22 |
| | | ID NO: 3 | 24 | h | 3.61 | 0.01 | 5.98 |
| | | 0.46% SEQ | 2 | h | 3.46 | 0.97 | 5.51 |
| | | ID NO: 3 | 24 | h | 3.22 | 1.49 | 5.74 |
| | 10% Glycerol | 0.1% SEQ | 2 | h | 3.58 | 0.80 | 5.69 |
| | | ID NO: 3 | 24 | h | 1.85 | 4.78 | 5.83 |
| | | 0.46% SEQ | 2 | h | 2.72 | 2.66 | 5.00 |
| | | ID NO: 3 | 24 | h | 3.44 | 1.34 | 5.46 |

From Table 8 and 9, FFA levels are well below 0.5% after just 2 h reaction in many cases, and even below 0.2% in some. After 2 h reaction time, TAG is not converted materially. But with 24 h reaction time CALB does convert up to 7% of the TAG at certain conditions, judging from the sum of measured components in the tables. The setup is competitive in pretreatment of eg. CPO for chemical biodiesel production, because of the extremely fast reaction of just 2 hours and comparatively low methanol dosage relative to the acid-catalyzed esterification method used industrially today.

Additionally, with the amount of glycerol added, which is not consumed, and which might even increase due to transesterification of MG and DG, CALB enzyme will be recyclable thereby further lowering the cost relative to currently employed industrial methods. This means a very low dosage will likely be sufficient for more environmentally friendly and economical substitution of the currently employed processes.

### Example 8: Performance with Esterase of SEQ ID NO: 4

CPO (4.4 wt % FFA, 6.1 wt % DAG) oil is melted at 60 C. 30 g CPO is then weighed off in 100 ml square bottles, followed by addition of 0 or 2% (wt/wt of oil) water and 0 or 5% (wt/wt of oil) dry technical grade glycerol. The mixture is conditioned in a shaking incubator oven at 40 C under 250 rpm mixing for 30 minutes to ensure the temperature is reached. 100 ppm NaOH is added as 50% aqueous solution.

An amount of active enzyme protein of SEQ ID NO: 4 equivalent to 0.2% (wt/wt of oil) SEQ ID NO: 1 is added at timepoint 0 h. Dry powder of the lipase was dissolved and added as 101 uL aqueous solution to the 30 g CPO, meaning an unavoidable and small amount of water was added with the lipase.

Reaction runs at 40 C in the shaking incubator at 250 rpm under 10 or 20 h continuous linear dosing of 1.7 molar equivalents of methanol based on free and bound fatty acids. After methanol dosing completion at the 10 or 20 h mark, reaction is continued until the 24 h mark and the results are measured.

2 mL samples are taken by pipette into 2 mL Eppendorf tubes. They are immediately centrifuged at 2000 rpm for 1 minute, before being incubated for 10 minutes at 99 C to inactivate the enzyme. Finally, the samples are dried under vacuum at 80 C for 2 hours to remove methanol.

### Methods for Analysis:

FFA by AOCS Official Method Ca 5a-40.
Mono- di, and triglycerides by AOCS Official Method Ck 2-09 using the Quality Trait Analysis (QTA) NIR method offered by Eurofins.

TABLE 10

| | | MG, | DG, | TG, | FFA, |
|---|---|---|---|---|---|
| 24 h reaction results | | wt % | wt % | wt % | wt % |
| 1.7 eqv. | 0% Glycerol | 0.784 | 0.323 | 0.13 | 1.03 |
| MeOH/10 h | 2% H20 | | | | |
| | 5% Glycerol | 0.87 | 1.022 | 1.185 | 0.43 |
| | 0% H20 | | | | |
| 1.7 eqv. | 0% Glycerol | 1.19 | 0.577 | 0.267 | 1.05 |
| MeOH/20 h | 2% H20 | | | | |
| | 5% Glycerol | 0.736 | 0.415 | 0.259 | 0.41 |
| | 0% H20 | | | | |

Considering the results using a 20 h dosage time, which are comparable to the previous examples, the results show that with 5% glycerol added instead of 2% water, there is an improvement in glyceride levels as the resulting as well FFA concentration.

The methanol-tolerant lipase yields a much better result with a 10 h methanol dosage time than would be obtained using SEQ ID NO: 1. The faster methanol dosage, when accompanied by added water, results in a faster reaction considering the concentration of glycerides. Further, when comparing the results of 10 h and 20 h dosage times using 5% glycerol instead of 2% water, the enzyme is markedly slower, and therefore it is clear that even the esterase of SEQ ID NO: 4 is negatively affected by methanol if too much is dosed too fast.

Finally, comparing these results to the previous examples, the final FFA concentrations are similar and largely correlate to the amount of water added with no further quality improvement provided by this very methanol-tolerant and fast lipase. That is despite the major difference in rate of reaction and possibility of fast methanol dosage when using SEQ ID NO: 4 rather than SEQ ID NO: 1. Evidently, reaction is equilibrium limited, and choice of lipase simply determines rate of reaction rather than product quality, when using a sufficiently methanol-tolerant lipase.

### Example 9: Glycerol Dosage Effect on SEQ ID NO: 4

CPO (4.4 wt % FFA, 6.1 wt % DAG) oil is melted at 60 C. 30 g CPO is then weighed off in 100 mL square bottles, followed by addition of 5, 10 or 20% (wt/wt of oil) dry technical grade glycerol. The mixture is conditioned in a shaking incubator oven at 40 C under 250 rpm mixing for 30 minutes to ensure the temperature is reached.

An amount of active enzyme protein of SEQ ID NO: 4 equivalent to 0.2% (wt/wt of oil) E SEQ ID NO: 1 is added at timepoint 0 h. Dry powder of the lipase was dissolved and added as 101 uL aqueous solution to the 30 g CPO, meaning an unavoidable and small amount of water was added with the lipase.

Reaction runs at 40 C in the shaking incubator at 250 rpm under 20 h continuous linear dosing of 1.7, 2 or 2.3 molar equivalents of methanol based on free and bound fatty acids. After methanol dosing completion at the 20 h mark, reaction is continued until the 24 h mark and the results are measured.

2 mL samples are taken by pipette into 2 mL Eppendorf tubes. They are immediately centrifuged at 2000 rpm for 1 minute, before being incubated for 10 minutes at 99 C to inactivate the enzyme. Finally, the samples are dried under vacuum at 80 C for 2 hours to remove methanol.

### Methods for Analysis:

FFA by AOCS Official Method Ca 5a-40.
Mono- di, and triglycerides by AOCS Official Method Ck 2-09 using the Quality Trait Analysis (QTA) NIR method offered by Eurofins.

TABLE 11

| | | MG, | DG, | TG, | FFA, |
|---|---|---|---|---|---|
| 24 h reaction | | wt % | wt % | wt % | wt % |
| 1.7 Eqv | 5% Glycerol | 0.76 | 0.38 | 0.27 | 0.46 |
| MeOH/20 h | 10% Glycerol | 0.85 | 0.42 | 0.29 | 0.42 |
| | 20% Glycerol | 0.76 | 0.36 | 0.24 | 0.35 |
| 2.0 Eqv | 5% Glycerol | 1.05 | 0.72 | 0.51 | 0.45 |
| MeOH/20 h | 10% Glycerol | 0.87 | 0.47 | 0.34 | 0.39 |
| | 20% Glycerol | 0.78 | 0.30 | 0.16 | 0.25 |
| 2.3 Eqv | 5% Glycerol | 0.84 | 0.77 | 0.57 | 0.47 |
| MeOH/20 h | 10% Glycerol | 0.73 | 0.50 | 0.30 | 0.35 |
| | 20% Glycerol | 0.82 | 0.54 | 0.47 | 0.45 |

Performance, especially on FFA levels, generally improves with more added glycerol. This is likely because the chemical activity of water decreases markedly in glycerol¹, meaning glycerol has an additional effect of effectively drying the reaction through its increased concentration. Notably, the combination of 2 equivalents of methanol along with 20% glycerol yields 0.25 wt % FFA, which is the target FFA level. Glyceride levels are generally higher than biodiesel specification levels, but reaction has only run for 24 h, and further glyceride conversion is expectable with more reaction time.

The differences between the results of 1.7 and 2.3 equivalents of methanol are small, and suggest water and glycerol are much more impactful parameters.

### Example 10: Obtaining <0.4 wt % MG after Reaction by Washing

CPO (4.4 wt % FFA, 6.1 wt % DAG) oil was used without pre-drying. 300 g of the CPO was then weighed off and added to a 1 L agitator-stirred glass reactor followed by addition of 20% (wt/wt of oil) dry technical grade glycerol. The mixture was conditioned at 35° C. and 400 rpm agitator speed for 30 minutes to ensure the temperature is reached. 0.5% (wt/wt of oil) of enzyme of SEQ ID NO: 1 was added at timepoint 0 h. Reaction runs at 35° C. in the reactor at 400 rpm under 40 h continuous linear dosing of 2 molar equivalents of methanol based on free and bound fatty acids. After methanol dosing completion at the 40 h mark, reaction was continued until the 72 h mark and the results are measured (0.4 wt % FFA, 0.72 wt % MAG and 0.11 wt % DAG was measured). This material was then used in the various washings that follows.

Washing: 10 g of the premade near-specifications FAME was added to a 25 mL plastic centrifuge tube. Then the washing mixture of choice (see the results table below) was added. Additives are premixed into the washing water where applicable. First the mixture is mixed by hand, followed by 2 minutes in ultrasonication bath at 60 C. Finally, the phases are separated by thorough centrifugation and the washed oil phase is sampled with resulting FFA, MAG, DAG and TAG values in Table 12.

For all samplings, 2 mL samples are taken by pipette into 2 mL Eppendorf tubes. They are immediately centrifuged at 2000 rpm for 1 minute, before being incubated for 10 minutes at 99 C to inactivate the enzyme. Finally, the samples are dried under vacuum at 80 C for 2 hours to remove methanol.

### Methods for Analysis:

FFA by AOCS Official Method Ca 5a-40. Mono- di, and triglycerides by AOCS Official. Method Ck 2-09 using the Quality Trait Analysis (QTA) NIR method offered

TABLE 12

| Water addition | Other | FFA | MAG | DAG | TAG |
|---|---|---|---|---|---|
| during washing | additives | (wt %) | (wt %) | (wt %) | (wt %) |
| Before Wash | | 0.40 | 0.72 | 0.11 | 0.00 |
| 2% (wt/wt oil) | 1 molar | 0.00 | 0.32 | 0.00 | 0.00 |
| Water added | equivalents of | | | | |
| | NaOH to FFA | | | | |
| 5% (wt/wt oil) | 1 molar | 0.00 | 0.32 | 0.00 | 0.00 |
| Water added | equivalents of | | | | |
| | NaOH to FFA | | | | |
| 2% (wt/wt oil) | 5 molar | 0.00 | 0.23 | 0.00 | 0.00 |
| Water added | equivalents of | | | | |
| | NaOH to FFA | | | | |
| 5% (wt/wt oil) | 5 molar | 0.00 | 0.32 | 0.00 | 0.00 |
| Water added | equivalents of | | | | |
| | NaOH to FFA | | | | |
| 2% (wt/wt oil) | 10 wt % Citric | 0.39 | 0.32 | 0.09 | 0.00 |
| Water added | Acid in the | | | | |
| | washing water | | | | |
| 2% (wt/wt oil) | 10 wt % NaCl | 0.64 | 0.26 | 0.08 | 0.00 |
| Water added | in the | | | | |
| | washing water | | | | |
| 2% (wt/wt oil) | — | 0.38 | 0.36 | 0.07 | 0.04 |
| Water added | | | | | |
| 5% (wt/wt oil) | — | 0.66 | 0.31 | 0.06 | 0.05 |
| Water added | | | | | |
| No water added | 1% silica | 0.38 | 0.36 | 0.08 | 0.07 |
| | (Sipernate | | | | |
| | S22) | | | | |

FFA levels of 0.4 wt % FFA rather than <0.25 wt % after initial reaction comes from lack of drying of the feedstock CPO. The FFA increases in two tests above, from 0.4 to 0.64 and 0.65, and suggest a small amount of active esterase was present in the feedstock oil after separation, and lead to hydrolysis with presence of water. However, the main results to consider in this example are the changes in MAG levels.

MAG reduction by residual FFA saponification was examined because it is conceivable that soaps could assist emulsification and thus MAG reduction. The effect of pH reduction and chelation by citric acid was examined, because low pH might have led to surprising results. The effect of high ionic strength through salt addition was examined, because this might have impacted the attraction of the hydrophilic parts of MAGs towards the water phase. And the effect of pure water (high purity, MilliQ) itself was measured as a baseline. Finally, hydrophilic, and small silica particles were tested as a possibility for adsorption of MAG and later filtering out.

MAG levels were comfortably reduced to below 0.4 wt % in all cases, suggesting the intrinsic oil-water emulsification property of MAGs is generally useful for washing out of this minor component, in cases where MAG levels would exceed desired levels.

Washing of finished biodiesel is a standard method in existing chemical biodiesel plants today, because soap is a byproduct of that process and is washed out by water. And in a state-of-the-art enzymatic biodiesel plant today, an alkaline washing step is also used today to saponify residual FFA. So a simple water-washing step is not introducing anything new to existing plants, rather such a washing step would likely produce a cleaner effluent than with existing processes, leading to reduced waste water treatment costs. And the losses during washing in both existing processes are significant, while the loss of this optional MAG-removal washing step would be less, and the MAG likely recyclable after possible separation from the washing water. Such separation could e.g. be through settling at high temperature and optionally low pH to break the MAG-water emulsion, or through solvent (biodiesel) extraction. Washing water might also be recyclable, with accumulation of MAG in a washing water settling tank, from which the MAG could then be recycled for better total process yield.

### Example 11: Biodiesel Reaction Using Two Enzymes with Medium to Poor Methanol Stability

Crude palm oil (CPO) oil without measured composition was melted at 60° C. 30 g of CPO was then weighed off in 100 mL square bottle. 50 ppm NaOH was added as 50% aqueous solution. The NaOH addition was determined from a standard curve previously made on the oil. The mixture was conditioned in a shaking incubator oven at 35° C. under 250 rpm mixing for 30 minutes to ensure that temperature was reached. At timepoint 0, the enzyme was added.

2.9 mg of esterase (SEQ ID NO: 5 or SEQ ID NO: 6) was added as premade esterase and glycerol mixtures. SEQ ID NO: 5 and SEQ ID NO: 6 originally had concentrations of 1.01 and 0.50 mg/mL, respectively, determined by standard A280 methods, meaning around 10% water would be dosed with the enzymes. Therefore, the esterase were dosed into the intended 20% (wt/wt) glycerol and dried for 10 h at 5.1 mbar and ambient temperature to remove water.

Reaction was performed at 35° C. in the shaking incubator at 250 rpm under 40 h continuous linear dosing of 2.0 molar equivalents of methanol based on an assumption of 100% triglyceride in the CPO. This meant a slight inaccuracy in dosage relative to true molar equivalents when accounting for concentrations of FFA, MG and DG in the oil, which was unknown for this particular oil. After methanol dosing completion at the 40 hours mark, reaction was continued until the 120 hours mark, after which reaction would have reached equilibrium and the results were measured.

2 mL samples are taken by pipette into 2 mL Eppendorf tubes. Sample was incubated for 10 minutes at 99° C. to inactivate the enzyme before being centrifuged at 2000 rpm for 1 minute. Finally, the samples are dried under vacuum at 80° C. for 2 hours to remove the excess methanol dissolved in the FAME.

FFA by AOCS Official Method Ca 5a-40. FAME, Mono- di, and triglycerides by AOCS Official Method Ck 2-09 using the Quality Trait Analysis (QTA) NIR method offered by Eurofins.

TABLE 13
Light phase biodiesel product components

| | Time | FAME | MG | DG | TG | FFA |
|---|---|---|---|---|---|---|
| Enzyme | [h] | [wt %] | [wt %] | [wt %] | [wt %] | [wt %] |
| SEQ ID | 24 | 78.82 | 5.096 | 4.595 | 8.192 | 3.07 |
| NO: 5 | 44 | 91.88 | 3.315 | 2.453 | 2.723 | 1.8 |
| | 48 | 91.31 | 3.387 | 1.776 | 2.706 | 1.92 |
| | 120 | 92.87 | 2.797 | 1.834 | 2.315 | 1.42 |
| SEQ ID | 24 | 80.81 | 4.079 | 3.408 | 6.46 | 1.58 |
| NO: 6 | 44 | 87.9 | 2.86 | 2.154 | 4.026 | 1.08 |
| | 48 | 87.78 | 2.943 | 1.76 | 3.938 | 1.12 |
| | 120 | 89.2 | 3.317 | 2.104 | 4.028 | 1.08 |

SEQ ID NO: 5 or SEQ ID NO: 6 performed well during the first 24 h of reaction, at which point 1.2 equivalents of methanol would have been added to the reaction. At the end of the methanol dosage regimen at 40 h, where 2 equivalents of methanol would have been added, reaction stalled, and poor additional conversion was achieved after additional reaction time. This example illustrates the fact that enzymes, which do not have sufficient stability, especially regarding tolerance of methanol in low-water environments, will not be able to utilize the invention to its full extent, because equilibrium is unobtainable with such enzymes due to premature inactivation. SEQ ID 1 and 4 are examples of enzymes with excellent tolerance of methanol, SEQ ID 4 in particular has excellent methanol tolerance. And such sufficient alcohol tolerance of a chosen enzyme is a prerequisite for employment of the invention with any chosen alcohol.

1-15. (canceled)

16. A process for reducing level of free fatty acids in biodiesel/fatty acid alkyl esters, said process comprising steps of:
(i) providing a fatty acid feedstock substrate comprising triglycerides, diglycerides, monoglycerides, free fatty acids, fatty acid esters, or any combination thereof;
(ii) reacting said fatty acid feedstock substrate with alcohol in the presence of an esterase and glycerol to produce fatty acid alkyl esters;
(iii) separating the reaction mixture of step (ii) into light phase comprising fatty acid methyl ester (FAME) and a heavy phase comprising esterase, glycerol, short chain alcohol and water;
(iv) drying the mixture of step (ii) followed by separation of the light and heavy phases; and
(v) drying the heavy phase of step (iii) in presence of the esterase, wherein the glycerol and esterase of the heavy phase is recycled and added back to step (ii);
wherein the resulting level of free fatty acid (FFA) concentration in the resulting biodiesel (FAME) product is below 1% (wt/wt).

17. The process according to claim 16, wherein the free fatty acid (FFA) concentration is below 1 wt % in the light phase of step iii) after removal of methanol from the light phase of step iii).

18. The process according to claim 16, wherein said esterase in step ii) is not immobilized.

19. The process according to claim 16, wherein said esterase in step ii) is added as a liquid, granule, and/or powder.

20. The process according to claim 16, wherein water added in step ii) is less than 2% wt/wt of the fatty acid feedstock.

21. The process according to claim 16, wherein the esterase is capable of transesterification of monoglycerides (MG), diglycerides (Dg), and triglycerides (TG).

22. The process according to claim 16, wherein the esterase comprises an amino acid sequence having at least 80% sequence identity to SEQ ID NO: 1.

23. The process according to claim 16, wherein the esterase comprises an amino acid sequence having at least 90% sequence identity to SEQ ID NO: 1.

24. The process according to claim 16, wherein the esterase comprises an amino acid sequence having at least 95% sequence identity to SEQ ID NO: 1.

25. The process according to claim 16, wherein the esterase comprises the amino acid of SEQ ID NO: 1.

26. The process according to claim 16, wherein said alcohol is a C1-C5 alcohol.

27. The process according to claim 16, wherein the process is performed at 20-90° C.

28. The process according to claim 16, wherein the substrate is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and animal fat.

29. The process according to claim 16, wherein the FFA is reduced with conversion of triglyceride being less than 30%.

30. The process according to claim 16, wherein the esterase comprises an amino acid sequence having at least 80% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

31. The process according to claim 16, wherein the esterase comprises an amino acid sequence having at least 90% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

32. The process according to claim 16, wherein the esterase comprises an amino acid sequence having at least 95% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

33. The process according to claim 16, wherein the esterase comprises the amino acid of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.
